(19) United States
(12) Patent Application Publication
(10) Pub. No.: US 2014/0088062 A1
(43) Pub. Date: Mar. 27, 2014

(54) COMPOSITIONS COMPRISING FUSIDIC ACID AND PACKAGES THEREFOR

(71) Applicant: David E. Pereira, Apex, NC (US)

(72) Inventor: David E. Pereira, Apex, NC (US)

(73) Assignee: CEM-102 PHARMACEUTICALS, INC., Chapel Hill, NC (US)

(21) Appl. No.: 14/119,542

(22) PCT Filed: May 23, 2012
(86) PCT No.: PCT/US12/39219

§ 371 Date: Nov. 22, 2013

Related U.S. Application Data
(60) Provisional application No. 61/489,017, filed on May 23, 2011.

(51) Int. Cl. A61K 47/10; A61J 1/00; A61K 31/575

(52) U.S. Cl. CPC A61K 47/10; A61K 31/575; A61J 1/00
USPC 514/182; 206/213.1

(57) ABSTRACT

Described are solid pharmaceutical compositions of fusidic acid, and pharmaceutically acceptable salts thereof, dosage units of the pharmaceutical compositions, and packages for pharmaceutical compositions of fusidic acid, and pharmaceutically acceptable salts thereof, which increase stability against the degradation of the fusidic acid, and pharmaceutically acceptable salts thereof. Also described are uses of the pharmaceutical compositions and dosage units in treating diseases.

### CROSS-REFERENCE TO RELATED APPLICATIONS

This application claims priority under 35 U.S.C. §119(e) to U.S. Provisional Application Ser. No. 61/489,017 filed May 23, 2011. The entire disclosure of which is incorporated herein by reference.

### TECHNICAL FIELD

The invention described herein pertains to solid pharmaceutical compositions of fusidic acid, and pharmaceutically acceptable salts thereof, dosage units of the pharmaceutical compositions, and packages for pharmaceutical compositions of fusidic acid, and pharmaceutically acceptable salts thereof. The invention described herein also pertains to solid pharmaceutical compositions and packages that may enhance stability to the degradation of the fusidic acid, or pharmaceutically acceptable salt thereof. The invention described herein also pertains to uses of the pharmaceutical compositions and dosage units in treating diseases.

### BACKGROUND AND SUMMARY OF THE INVENTION

Fusidic acid is a tetracyclic triterpenoid or fusidane antibiotic derived from the fungus Fusidium coccineum that inhibits bacterial protein synthesis. Fusidic Acid has the following structure.

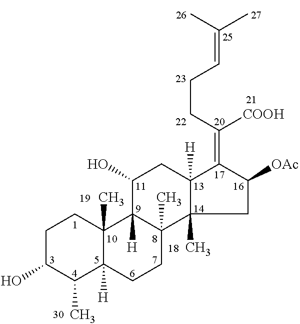

The term fusidic acid is also often used to denote not only fusidic acid, but also its pharmaceutically acceptable salts, such as the sodium salt, sodium fusidate, as well as hydrates, solvates or mixtures thereof, any of which may be considered the fusidic acid component. Accordingly, as used herein, the term fusidic acid generally refers individually and collectively to fusidic acid itself, salts of fusidic acid, certain hydrolyzable esters thereof, and salts of such esters, which may serve as prodrugs, as well as modified fusidic acid derivatives, such as the 24,25-dihydro and 17,20-methano derivatives and the pharmaceutically acceptable salts and easily hydrolyzable esters thereof. Generally, the foregoing are also referred to as fusidates. Fusidic acid, fusidates, and the like have been administered for the treatment of a variety of bacterial infections. However, the full potential of fusidic acid, or other fusidates, in treating diseases is hampered by the lack of long-term storage properties of the compounds.

It has been unexpectedly discovered herein that oxidative degradation is a primary degradation pathway of fusidic acid and fusidates, pharmaceutically acceptable salts of fusidic acid and fusidates, and compositions comprising fusidic acid and fusidates, and/or pharmaceutically acceptable salts of fusidic acid and fusidates. Without being bound by theory, it is believed herein that such oxidative degradation contributes to the lack of long-term storage properties of the compounds. In addition, but without being bound by theory, it is believed herein that such oxidative degradation is caused by ambient and/or atmospheric oxygen.

It has also been unexpectedly discovered herein that hydrolytic degradation, or hydrolysis, is a primary degradation pathway of fusidic acid and fusidates, pharmaceutically acceptable salts of fusidic acid and fusidates, and compositions comprising fusidic acid and fusidates, and/or pharmaceutically acceptable salts of fusidic acid and fusidates. Without being bound by theory, it is believed herein that such hydrolytic degradation, or hydrolysis, contributes to the lack of long-term storage properties of the compounds. In addition, but without being bound by theory, it is believed herein that such hydrolytic degradation, or hydrolysis, is caused by ambient and/or atmospheric water, and/or water inherently in the composition.

It has also been discovered that fusidic acid, pharmaceutically acceptable salts of fusidic acid, and compositions comprising fusidic acid and/or pharmaceutically acceptable salts of fusidic acid may be stabilized to provide a longer storage life with lower degradation of active pharmaceutical ingredient (API).

In one embodiment, fusidic acid, pharmaceutically acceptable salts of fusidic acid, and/or compositions comprising fusidic acid and/or pharmaceutically acceptable salts of fusidic acid are described that include a component of excipient that is capable of decreasing the amount of degradation of the fusidic acid component, or salt thereof. In another embodiment, fusidic acid, pharmaceutically acceptable salts of fusidic acid, and/or compositions comprising fusidic acid and/or pharmaceutically acceptable salts of fusidic acid are described that include mannitol. It has been unexpectedly discovered that mannitol is capable of decreasing the amount of degradation of the fusidic acid API.

In another embodiment, packages for fusidic acid, pharmaceutically acceptable salts of fusidic acid, and/or compositions comprising fusidic acid and/or pharmaceutically acceptable salts of fusidic acid are described that are capable of decreasing the amount of degradation of the fusidic acid component, or salt thereof. In another embodiment, packages containing fusidic acid, pharmaceutically acceptable salts of fusidic acid, and/or compositions comprising fusidic acid and/or pharmaceutically acceptable salts of fusidic acid are described, where the fusidic acid is in API form, or alternatively included in a solid unit dosage form. The packaged fusidic acid component, or salt thereof is stabilized to degradation.

### BRIEF DESCRIPTION OF THE DRAWINGS

FIG. 1 shows the percent of initial assay of 600 mg tablets in HDPE at 25° C./60% RH for formulation 44I compared to formulation 44F.

FIG. 2 shows the percent of initial assay of 600 mg tablets in HDPE at 40° C./75% RH for formulation 44I compared to formulation 44F.

FIG. 3. shows the percent change in 27-oxofusidic acid in 600 mg tablets in HDPE or O₂ resistant HDPE at 25° C./60% RH.

FIG. 4 shows the percent change in the total of 3-ketofusidic acid and 11-ketofusidic acid in 600 mg tablets in HDPE or O₂ resistant HDPE at 25° C./60% RH.

FIG. 5 shows the percent change in 27-oxofusidic acid in 300 mg tablets in HDPE or HDPE/Stabilox at 25° C./60% RH.

FIG. 6 shows the percent change in the total of 3-ketofusidic acid and 11-ketofusidic acid in 300 mg Tablets in HDPE or HDPE/Stabilox at 25° C./60% RH.

### DETAILED DESCRIPTION

Described herein are compositions, formulations, processes, and packages that increase the storage stability of fusidic acid, including solid dosage forms, such as tablets, comprising fusidic acid. Also described herein are compositions, formulations, processes, and packages that decrease the number of, amount of, and/or the rate of formation of impurities during the storage of fusidic acid, including solid dosage forms, such as tablets, comprising fusidic acid. Also described herein are compositions, formulations, processes, and packages that decrease the number of, amount of, and/or the rate of formation of impurities by oxidation during the storage of fusidic acid, including solid dosage forms, such as tablets, comprising fusidic acid. Also described herein are compositions, formulations, processes, and packages that decrease the number of, amount of, and/or the rate of formation of impurities by hydrolysis during the storage of fusidic acid, including solid dosage forms, such as tablets, comprising fusidic acid.

In one embodiment, solid formulations of fusidic acid, and pharmaceutically acceptable salts thereof, are described herein, where the solid formulations maintain an assay upon prolonged storage of at least about 90%, least about 91%, least about 92%, least about 93%, least about 94%, at least about 95%, at least about 96%, at least about 97%, at least about 98%, at least about 99%, at least about 99.5%, at least about 99.7%, or at least about 99.8%, compared to the initial assay. Prolonged storage includes storage times of about 3 months, about 6 months, about 9 months, about 12 months, about 18 months, and/or about 24 months.

In another embodiment, solid formulations of fusidic acid, and pharmaceutically acceptable salts thereof, are described herein, where the solid formulations maintain an assay upon prolonged storage of at least about 90%, least about 91%, least about 92%, least about 93%, least about 94%, at least about 95%, at least about 96%, at least about 97%, at least about 98%, at least about 99%, at least about 99.5%, or at least about 99.8%, compared to the initial assay. Prolonged storage includes storage temperatures of about ambient, about 25° C., about 30° C., about 35° C., or about 40° C.

In another embodiment, solid formulations of fusidic acid, and pharmaceutically acceptable salts thereof, are described herein, where the solid formulations maintain an assay upon prolonged storage of at least about 90%, least about 91%, least about 92%, least about 93%, least about 94%, at least about 95%, at least about 96%, at least about 97%, at least about 98%, at least about 99%, at least about 99.5%, or at least about 99.8%, compared to the initial assay. Prolonged storage includes storage humidity of about 60%, about 65%, about 70%, or about 75%.

In another embodiment, solid formulations of fusidic acid, and pharmaceutically acceptable salts thereof, are described herein, where the solid formulations maintain an assay level after prolonged storage of at least about 90%, 91%, 92%, 93%, 94%, or 95%, as compared to the initial assay. Prolonged storage includes storage times, storage temperatures, and storage humidity, as described herein. Illustratively, prolonged storage includes 25° C./60% RH for 1 year, 25° C./60% RH for 1.5 years, or 25° C./60% RH for 2 years; or 40° C./75% RH for 6 months, or 40° C./75% RH for 12 months.

In another embodiment, solid formulations of fusidic acid, and pharmaceutically acceptable salts thereof, are described herein, where the solid formulations maintain an impurity increase upon prolonged storage of less than about 3-fold, less than about 2.5-fold, less than about 2-fold, less than about 50%, less than about 25%, less than about 10%, less than about 5%, or less than about 2%. Prolonged storage includes storage times, storage temperatures, and storage humidity, as described herein. Illustratively, prolonged storage includes 25° C./60% RH for 2 years.

Illustrative impurities include one or more of the following:

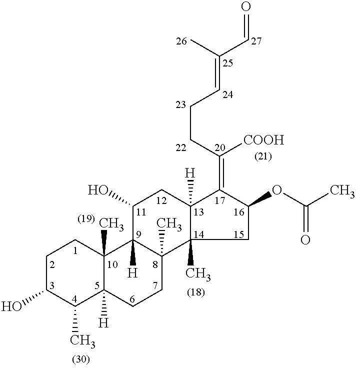

also referred to as 27-oxofusidic acid (compound F),

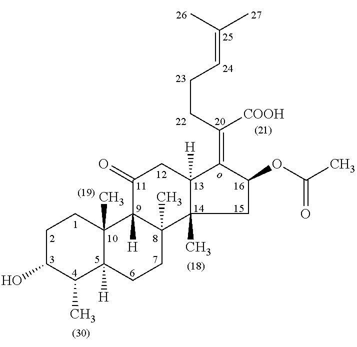

also referred to as 11-ketofusidic acid (compound H),

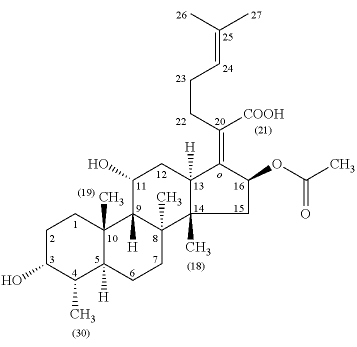

also referred to as 3-ketofusidic acid (compound G),

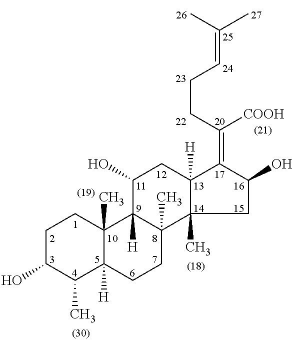

also referred to as 16-desacetylfusidic acid (compound O),

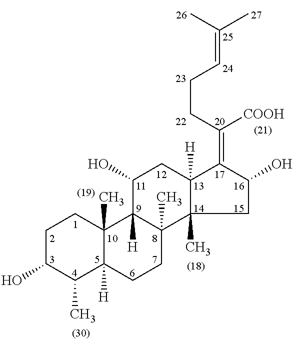

also referred to as epi-16-desacetylfusidic acid (compound I), and

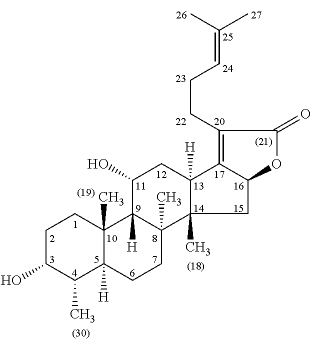

also referred to as 16-desacetylfusidic acid-21,16-lactone (compound K). It is to be understood that depending upon the assay method, measurement method, or other analytical evaluation, 16-desacetylfusidic acid (compound O) may convert to 16-desacetylfusidic acid-21,16-lactone (compound K) during the analysis. Therefore, it is to be understood that as described herein, the amount of, or percentage change in, compound K is understood to include the total of compound K and compound O, when the analysis results in the conversion of compound O to compound K.

In another embodiment, solid formulations of fusidic acid, and pharmaceutically acceptable salts thereof, are described herein, where the solid formulations exhibit a lower amount of, and/or a slower rate of formation of one or more of 27-oxofusidic acid, 11-ketofusidic acid, and 3-ketofusidic acid during the storage of fusidic acid.

In another embodiment, solid formulations of fusidic acid, and pharmaceutically acceptable salts thereof, are described herein, where the solid formulations exhibit a lower amount of, and/or a slower rate of formation of one or more of 16-desacetylfusidic acid, epi-16-desacetylfusidic acid, or 16-desacetylfusidic acid-21,16-lactone during the storage of fusidic acid.

In another embodiment, solid formulations of fusidic acid, or pharmaceutically acceptable salts thereof, and packaged articles containing such solid formulations are described herein, where the solid formulations exhibit after prolonged storage a total amount of 27-oxofusidic acid that is less than about 0.2%, or less than about 0.15%. In another embodiment, solid formulations of fusidic acid, or pharmaceutically acceptable salts thereof, and packaged articles containing such solid formulations are described herein, where the solid formulations exhibit after prolonged storage a total amount of 11-ketofusidic acid that is less than about 0.2%, or less than about 0.15%. In another embodiment, solid formulations of fusidic acid, or pharmaceutically acceptable salts thereof, and packaged articles containing such solid formulations are described herein, where the solid formulations exhibit after prolonged storage a total amount of 3-ketofusidic acid that is less than about 0.2%, or less than about 0.15%. In another embodiment, solid formulations of fusidic acid, or pharmaceutically acceptable salts thereof, and packaged articles containing such solid formulations are described herein, where the solid formulations exhibit after prolonged storage a total amount of 16-desacetylfusidic acid that is less than about 0.2%, or less than about 0.15%. In another embodiment, solid formulations of fusidic acid, or pharmaceutically acceptable salts thereof, and packaged articles containing such solid formulations are described herein, where the solid formulations exhibit after prolonged storage a total amount of epi-16-desacetylfusidic acid that is less than about 0.2%, or less than about 0.15%. In another embodiment, solid formulations of fusidic acid, or pharmaceutically acceptable salts thereof, and packaged articles containing such solid formulations are described herein, where the solid formulations exhibit after prolonged storage a total amount of 16-desacetylfusidic acid-21,16-lactone that is less than about 0.2%, or less than about 0.15%. Prolonged storage includes storage times, storage temperatures, and storage humidity, as described herein. Illustratively, prolonged storage includes 25° C./60% RH for 1 year, 25° C./60% RH for 1.5 years, or 25° C./60% RH for 2 years; or 40° C./75% RH for 6 months, or 40° C./75% RH for 12 months.

In another embodiment, solid formulations of fusidic acid, or pharmaceutically acceptable salts thereof, and packaged articles containing such solid formulations are described herein, where the solid formulations exhibit after prolonged storage a total amount of 27-oxofusidic acid that is less than about 2-fold higher, less than about 50% higher, or less than about 25% higher compared to the initial time. In another embodiment, solid formulations of fusidic acid, or pharmaceutically acceptable salts thereof, and packaged articles containing such solid formulations are described herein, where the solid formulations exhibit after prolonged storage a total amount of 11-ketofusidic acid that is less than about 2-fold higher, less than about 50% higher, or less than about 25% higher compared to the initial time. In another embodiment, solid formulations of fusidic acid, or pharmaceutically acceptable salts thereof, and packaged articles containing such solid formulations are described herein, where the solid formulations exhibit after prolonged storage a total amount of 3-ketofusidic acid that is less than about 2-fold higher, less than about 50% higher, or less than about 25% higher compared to the initial time. In another embodiment, solid formulations of fusidic acid, or pharmaceutically acceptable salts thereof, and packaged articles containing such solid formulations are described herein, where the solid formulations exhibit after prolonged storage a total amount of 16-desacetylfusidic acid that is less than about 2-fold higher, less than about 50% higher, or less than about 25% higher compared to the initial time. In another embodiment, solid formulations of fusidic acid, or pharmaceutically acceptable salts thereof, and packaged articles containing such solid formulations are described herein, where the solid formulations exhibit after prolonged storage a total amount of epi-16-desacetylfusidic acid that is less than about 2-fold higher, less than about 50% higher, or less than about 25% higher compared to the initial time. In another embodiment, solid formulations of fusidic acid, or pharmaceutically acceptable salts thereof, and packaged articles containing such solid formulations are described herein, where the solid formulations exhibit after prolonged storage a total amount of 16-desacetylfusidic acid-21,16-lactone that is less than about 2-fold higher, less than about 50% higher, or less than about 25% higher compared to the initial time. Prolonged storage includes storage times, storage temperatures, and storage humidity, as described herein. Illustratively, prolonged storage includes 25° C./60% RH for 1 year, 25° C./60% RH for 1.5 years, or 25° C./60% RH for 2 years; or 40° C./75% RH for 6 months, or 40° C./75% RH for 12 months. In addition, prolonged storage includes 25° C./60% RH for 3 months, 25° C./60% RH for 6 months, or 25° C./60% RH for 9 months; or 40° C./75% RH for 3 months.

In another embodiment, a solid pharmaceutical composition comprising fusidic acid, or a pharmaceutically acceptable salt thereof, and mannitol is described.

In another embodiment, a dosage unit comprising a solid pharmaceutical composition comprising fusidic acid, or a pharmaceutically acceptable salt thereof, and mannitol, where the fusidic acid or salt thereof is present in the range from about 250 mg to about 1,000 mg is described. In another embodiment, a dosage unit comprising a solid pharmaceutical composition comprising fusidic acid, or a pharmaceutically acceptable salt thereof, and mannitol, where the fusidic acid or salt thereof is present in the range from about 275 mg to about 1,000 mg is described. Another embodiment of the dosage unit is one where the fusidic acid or salt thereof is present in the range from about 300 mg to about 900 mg. Another embodiment of the dosage unit is one where the fusidic acid or salt thereof is present in the range from about 300 mg to about 800 mg. Another embodiment of the dosage unit is one where the fusidic acid or salt thereof is present in the range from about 300 mg to about 700 mg. Another embodiment of the dosage unit is one where the fusidic acid or salt thereof is present in the range from about 300 mg to about 600 mg. Another embodiment of the dosage unit is one where the fusidic acid or salt thereof is present at about 300 mg. Another embodiment of the dosage unit is one where the fusidic acid or salt thereof is present at about 600 mg.

Another embodiment of the above pharmaceutical composition or any of the above dosage units is one wherein the w/w ratio of the fusidic acid or salt thereof to mannitol is in the range from about 1:1 to about 10:1. Another embodiment of the above pharmaceutical composition or any of the above dosage units is one wherein the w/w ratio of the fusidic acid or salt thereof to mannitol is in the range from about 2:1 to about 5:1. Another embodiment of the above pharmaceutical composition or any of the above dosage units is one wherein the w/w ratio of the fusidic acid or salt thereof to mannitol is in the range from about 3:1 to about 4:1.

In another embodiment, a packaged article comprising a dosage unit comprising a solid pharmaceutical composition comprising fusidic acid, or a pharmaceutically acceptable salt thereof, where the fusidic acid or salt thereof is present in the range from about 250 mg to about 1,000 mg is described. In another embodiment, a packaged article comprising a solid pharmaceutical composition comprising fusidic acid, or a pharmaceutically acceptable salt thereof, where the fusidic acid or salt thereof is present in the range from about 275 mg to about 1,000 mg is described. Another embodiment of the packaged article is one where the fusidic acid or salt thereof is present in the range from about 300 mg to about 900 mg. Another embodiment of the packaged article is one where the fusidic acid or salt thereof is present in the range from about 300 mg to about 800 mg. Another embodiment of the packaged article is one where the fusidic acid or salt thereof is present in the range from about 300 mg to about 700 mg. Another embodiment of the packaged article is one where the fusidic acid or salt thereof is present in the range from about 300 mg to about 600 mg. Another embodiment of the packaged article is one where the fusidic acid or salt thereof is present at about 300 mg. Another embodiment of the packaged article is one where the fusidic acid or salt thereof is present at about 600 mg. In each of the foregoing packaged articles, the packaged articles further comprise one or more of (a) an oxygen resistant and/or oxygen impermeable container, and/or (b) an oxidant absorbent, and/or antioxidant compound or composition, and/or (c) an atmosphere with reduced oxygen and/or that is substantially free of oxygen.

Another embodiment of any of the above pharmaceutical compositions or any of the above dosage units is one wherein the fusidic acid or salt thereof is present at about 10% to about 90% by weight. Another embodiment of the above pharmaceutical compositions or any of the above dosage units is one wherein the fusidic acid or salt thereof is present at about 20% to about 80% by weight. Another embodiment of the above pharmaceutical compositions or any of the above dosage units is one wherein the fusidic acid or salt thereof is present at about 30% to about 70% by weight. Another embodiment of the above pharmaceutical compositions or any of the above dosage units is one wherein the fusidic acid or salt thereof is present at about 40% to about 60% by weight.

In another embodiment, the package is configured as a bulk dosage container. In another embodiment, the package is configured as an individual dosage container. In one variation, the package for the above pharmaceutical composition or dosage unit(s) is an oxygen resistant and/or oxygen impermeable container. Illustrative oxygen resistant and/or oxygen impermeable containers include, but are not limited to, oxygen resistant high-density polyethylene (HDPE) containers, laminated or layered plastic containers, such as a layered configuration having an ethylene vinyl alcohol (EVOH) layer sandwiched between two layers of HDPE, including StabilitySolutions™ Barrier containers available from Alcan, Oxy-Guard containers available from Süd-Chemie Performance Packaging (Belen, N. Mex.), TOPAS® COC and Multilayer PET containers available from TOPAS Advanced Polymers, Inc. (Florence, Ky.), polyethylene terephthalate (PET) containers, glass bottles, and the like. Illustrative high gas barrier plastic bottles are extrusion blow-molded with up to six layers and may provide a barrier up to 100 times more effective than conventional polyethylene. Another illustrative high barrier container is fabricated from an EVOH layer that is sandwiched between two layers of HDPE, available from Alcan. In each case, the containers may include a child-resistant (CR) cap.

Accordingly, a packaged article comprising a container and the above pharmaceutical composition or any of the above dosage units wherein the pharmaceutical composition or the dosage unit is inside the container and wherein the container is an oxygen resistant and/or oxygen impermeable container is described.

In one variation, the package for any of the above pharmaceutical composition or dosage unit(s) and containers includes an oxidant absorbent, antioxidant compound or composition. Illustrative oxidant absorbents, antioxidant compounds and compositions include, but are not limited to iron-containing absorbents, StabilOx™ packets (Healthcare Packaging Division, Multisorb Technologies), PharmaKeep® packets (Süd-Chemie Performance Packaging), OUKPAC (Nantong Ouk Packaging Engineering), O-Busters® Oxygen Absorbing Packets (Delta Absorbents), and the like, and combinations thereof. Accordingly, described herein is a packaged article comprising a closed or closeable container and the above pharmaceutical composition or any of the above dosage units wherein the pharmaceutical composition or the dosage unit is inside the container and wherein the container or package includes an absorbent, antioxidant compound or composition. In one embodiment, the container includes a StabilOx insert.

In another embodiment, the container comprises a polymer film attached to a metal foil, such as PVdC/250 PVC blister film with foil pouch overwrap with an oxygen absorbent and/or antioxidant compound or composition; PVdC/250 PVC blister film with foil pouch overwrap without absorbent. In another embodiment, the package is PVdC/250 PVC blister film with foil pouch overwrap without absorbent and nitrogen flushed; or PVdC/250 PVC blister film without foil pouch overwrap and nitrogen flushed. In another embodiment, the package is Mono 250 PVC blister film with foil pouch overwrap with an oxygen absorbent and/or antioxidant compound or composition. In another embodiment, the package is foil-foil blister pack that includes a foil film with foil pouch overwrap. Illustrative foil-foil blister packs are commercially available from Norsk Hydro ASA (Oslo, Norway). Comparison containers include, PVdC/250 PVC blister film without foil pouch overwrap.

In another variation, the package for any of the above pharmaceutical composition or dosage unit(s) and containers includes an atmosphere with reduced oxygen or alternatively an atmosphere that is substantially free of oxygen. Illustrative atmospheres include, but are not limited to, nitrogen, argon, and the like, and combinations thereof. Accordingly, a packaged article is described comprising a container and the above pharmaceutical composition or any of the above dosage units wherein the pharmaceutical composition or the dosage unit is inside the container and wherein the container or package includes an atmosphere with reduced oxygen or alternatively an atmosphere that is substantially free of oxygen. In one embodiment, the container or package includes a nitrogen atmosphere with reduced oxygen or free of oxygen.

In another variation, any of the foregoing package embodiments is configured for evacuation, such as vacuum packing. Vacuum packing may be performed by any conventional method or process, using any conventional apparatus. It is to be understood that vacuum packing is an alternative method for packaging in an atmosphere with reduced oxygen or alternatively an atmosphere that is substantially free of oxygen, where evacuation is complete or nearly complete. It is also to be understood that vacuum packing may be included as an initial step fro replacing ambient atmosphere with an atmosphere with reduced oxygen or alternatively an atmosphere that is substantially free of oxygen. Following evacuation, an atmosphere with reduced oxygen or alternatively an atmosphere that is substantially free of oxygen may be introduced.

In another variation, any of the foregoing package embodiments is configured to include a dehumidifying component, such as Tri-Sorb® packet and the like. It is to be understood that the dehumidifying component may be included with the oxidant absorbent.

Further, a packaged article is described comprising a container and the above pharmaceutical composition or any of the above dosage units wherein the pharmaceutical composition or the dosage unit is inside the container and wherein:

a) the container is an oxygen resistant and/or oxygen impermeable container and the container or package includes an antioxidant compound or composition; or

b) the container is an oxygen resistant and/or oxygen impermeable container and the container or package includes an atmosphere with reduced oxygen or that is substantially free of oxygen; or

c) the container or package includes an antioxidant compound or composition and the container or package includes an atmosphere with reduced oxygen or that is substantially free of oxygen.

Further, described herein is a packaged article comprising a container and the above pharmaceutical composition or any of the above dosage units wherein the pharmaceutical composition or the dosage unit is inside the container and wherein the container is an oxygen resistant and/or oxygen impermeable container; the container or package includes an antioxidant compound or composition; and the container or package includes an atmosphere with reduced oxygen or that is substantially free of oxygen.

In another variation, the package is an oxygen resistant and/or oxygen impermeable container, and includes an antioxidant compound or composition. In another variation, the package is an oxygen resistant and/or oxygen impermeable container, and includes an atmosphere with reduced oxygen or alternatively an atmosphere that is substantially free of oxygen. In another variation, the package includes an antioxidant compound or composition, and includes an atmosphere with reduced oxygen or alternatively an atmosphere that is substantially free of oxygen. In another variation, the package is an oxygen resistant and/or oxygen impermeable container, includes an antioxidant compound or composition, and includes an atmosphere with reduced oxygen or alternatively an atmosphere that is substantially free of oxygen.

A solid pharmaceutical composition comprising fusidic acid, or a pharmaceutically acceptable salt thereof, and mannitol may be prepared in a conventional manner. A dosage unit comprising a solid pharmaceutical composition comprising fusidic acid, or a pharmaceutically acceptable salt thereof, and mannitol; may be prepared in a conventional manner, including that disclosed in WO 96/03128.

It is to be understood that the fusidic acid, pharmaceutically acceptable salts of fusidic acid, and/or compositions comprising fusidic acid and/or pharmaceutically acceptable salts of fusidic acid that are stabilized by adding an excipient that is capable of decreasing the amount of oxidation of the fusidic acid component, or salt thereof, may be further stabilized by including one or more of the packages described herein. Accordingly, the stability of the API may be assayed for the pharmaceutical composition alone, for a dosage unit comprising the pharmaceutical composition, or for the pharmaceutical composition or the dosage unit comprising the pharmaceutical composition within a particular package.

One embodiment of the above pharmaceutical composition or any of the above dosage units or packages containing the pharmaceutical composition or dosage units is one wherein the fusidic acid or salt thereof decreases by about 10% or less after 2 years at 25° C. and 60% RH. Another such embodiment is one wherein the fusidic acid or salt thereof decreases by about 9% or less after 2 years at 25° C. and 60% RH. Another such embodiment is one wherein the fusidic acid or salt thereof decreases by about 8% or less after 2 years at 25° C. and 60% RH. Another such embodiment is one wherein the fusidic acid or salt thereof decreases by about 7% or less after 2 years at 25° C. and 60% RH. Another such embodiment is one wherein the fusidic acid or salt thereof decreases by about 6% or less after 2 years at 25° C. and 60% RH. Another such embodiment is one wherein the fusidic acid or salt thereof decreases by about 5% or less after 2 years at 25° C. and 60% RH. Another such embodiment is one wherein the fusidic acid or salt thereof decreases by about 10% or less after 1 year at 40° C. and 75% RH. Another such embodiment is one wherein the fusidic acid or salt thereof decreases by about 9% or less after 1 year at 40° C. and 75% RH. Another such embodiment is one wherein the fusidic acid or salt thereof decreases by about 8% or less after 1 year at 40° C. and 75% RH. Another such embodiment is one wherein the fusidic acid or salt thereof decreases by about 7% or less after 1 year at 40° C. and 75% RH. Another such embodiment is one wherein the fusidic acid or salt thereof decreases by about 6% or less after 1 year at 40° C. and 75% RH. Another such embodiment is one wherein the fusidic acid or salt thereof decreases by about 5% or less after 1 year at 40° C. and 75% RH.

Another such embodiment is one wherein the fusidic acid or salt thereof decreases by about 5% or less after 6 months at 40° C. and 75% RH. Another such embodiment is one wherein the fusidic acid or salt thereof decreases by about 4% or less after 6 months at 25° C. and 60% RH. Another such embodiment is one wherein the fusidic acid or salt thereof decreases by about 4% or less after 6 months at 40° C. and 75% RH. Another such embodiment is one wherein the fusidic acid or salt thereof decreases by about 3% or less after 6 months at 25° C. and 60% RH. Another such embodiment is one wherein the fusidic acid or salt thereof decreases by about 3% or less after 6 months at 40° C. and 75% RH. Another such embodiment is one wherein the fusidic acid or salt thereof decreases by about 2% or less after 6 months at 25° C. and 60% RH. Another such embodiment is one wherein the fusidic acid or salt thereof decreases by about 2% or less after 6 months at 40° C. and 75% RH. Another such embodiment one wherein the fusidic acid or salt thereof decreases by about 1% or less after 6 months at 25° C. and 60% RH. Another such embodiment is one wherein the fusidic acid or salt thereof decreases by about 1% or less after 6 months at 40° C. and 75% RH.

It is to be understood that if the fusidic acid or salt thereof (API) decreases, for example, by about 5% or less, the % API remaining will be the corresponding difference, for example, about 95% or greater, normalized to the starting time. It is also to be understood that as used herein, the term assay value generally corresponds to the fusidic acid or salt thereof that is present in, or remains in, the tested API, formulation, dosage unit, and the like.

In another embodiment of the above pharmaceutical composition or any of the above dosage units or packages containing the pharmaceutical composition or dosage units is one wherein the impurity level increases by about 3-fold or less or by about 2.5-fold or less after 6 months at 25° C. and 60% RH. Another such embodiment is one wherein the impurity level increases by about by about 3-fold or less or 2.5-fold or less after 6 months at 40° C. and 75% RH. In another embodiment of the above pharmaceutical composition or any of the above dosage units or packages containing the pharmaceutical composition or dosage units is one wherein the impurity level increases by about 100% or less after 6 months at 25° C. and 60% RH. Another such embodiment is one wherein the impurity level increases by about 100% or less after 6 months at 40° C. and 75% RH. In another embodiment of the above pharmaceutical composition or any of the above dosage units or packages containing the pharmaceutical composition or dosage units is one wherein the impurity level increases by about 50% or less after 6 months at 25° C. and 60% RH. Another such embodiment is one wherein the impurity level increases by about 50% or less after 6 months at 40° C. and 75% RH. In another embodiment of the above pharmaceutical composition or any of the above dosage units or packages containing the pharmaceutical composition or dosage units is one wherein the impurity level increases by about 25% or less after 6 months at 25° C. and 60% RH. Another such embodiment is one wherein the impurity level increases by about 25% or less after 6 months at 40° C. and 75% RH.

In another embodiment of the above pharmaceutical composition or any of the above dosage units or packages containing the pharmaceutical composition or dosage units is one wherein the impurity level increases by about 2.5-fold or less after 12 months at 25° C. and 60% RH. Another such embodiment is one wherein the impurity level increases by about 2.5-fold or less after 12 months at 40° C. and 75% RH. In another embodiment of the above pharmaceutical composition or any of the above dosage units or packages containing the pharmaceutical composition or dosage units is one wherein the impurity level increases by about 100% or less after 12 months at 25° C. and 60% RH. Another such embodiment is one wherein the impurity level increases by about 100% or less after 12 months at 40° C. and 75% RH. In another embodiment of the above pharmaceutical composition or any of the above dosage units or packages containing the pharmaceutical composition or dosage units is one wherein the impurity level increases by about 50% or less after 12 months at 25° C. and 60% RH. Another such embodiment is one wherein the impurity level increases by about 50% or less after 12 months at 40° C. and 75% RH. In another embodiment of the above pharmaceutical composition or any of the above dosage units or packages containing the pharmaceutical composition or dosage units is one wherein the impurity level increases by about 25% or less after 12 months at 25° C. and 60% RH. Another such embodiment is one wherein the impurity level increases by about 25% or less after 12 months at 40° C. and 75% RH.

In another embodiment of the above pharmaceutical composition or any of the above dosage units or packages containing the pharmaceutical composition or dosage units is one where the solid formulations have one or more of the following characteristics:

| | | Storage Time |
|---|---|---|
| 27-oxofusidic acid | Storage Condition | (Months) |
| less than about 0.2% | 25° C./60% RH | 12 |
| less than about 0.2% | 25° C./60% RH | 18 |
| less than about 0.2% | 25° C./60% RH | 24 |
| less than about 0.2% | 40° C./75% RH | 6 |
| less than about 0.2% | 40° C./75% RH | 9 |
| less than about 0.2% | 40° C./75% RH | 12 |

| | | Storage Time |
|---|---|---|
| Total of 3- and/or 11-ketofusidic acid | Storage Condition | (Months) |
| less than about 0.2% | 25° C./60% RH | 12 |
| less than about 0.2% | 25° C./60% RH | 18 |
| less than about 0.2% | 25° C./60% RH | 24 |
| less than about 0.2% | 40° C./75% RH | 6 |
| less than about 0.2% | 40° C./75% RH | 9 |
| less than about 0.2% | 40° C./75% RH | 12 |

| | | Storage Time |
|---|---|---|
| Total of epi-16-desacetylfusidic acid | Storage Condition | (Months) |
| less than about 0.2% | 25° C./60% RH | 12 |
| less than about 0.2% | 25° C./60% RH | 18 |
| less than about 0.2% | 25° C./60% RH | 24 |
| less than about 0.2% | 40° C./75% RH | 6 |
| less than about 0.2% | 40° C./75% RH | 9 |
| less than about 0.2% | 40° C./75% RH | 12 |

| Total of 16-desacetylfusidic acid-21, | | Storage Time |
|---|---|---|
| 6-lactone | Storage Condition | (Months) |
| less than about 0.2% | 25° C./60% RH | 12 |
| less than about 0.2% | 25° C./60% RH | 18 |
| less than about 0.2% | 25° C./60% RH | 24 |
| less than about 0.2% | 40° C./75% RH | 6 |
| less than about 0.2% | 40° C./75% RH | 9 |
| less than about 0.2% | 40° C./75% RH | 12 |

As used herein a dosage unit or unit dosage form generally refers to a tablet, capsule, suppository, ampoule, vial or other device, containing a definite amount of a drug, the whole of which is intended to be administered at a predetermined dosing event. It is to be understood that multiple dosage units may be administered at such a predetermined dosing event. The dosage units of the solid pharmaceutical compositions of the fusidic acid component may be prepared by conventional methods for the particular form of finished product. The dosage units may be packaged in a container containing a number of doses, in unit-dose packaging as one or more dosage units forming a single dose in a non-reusable container, or in single dosage unit containers.

In another embodiment, described herein is a packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, wherein the fusidic acid, salt thereof, or a combination thereof degrades by about 10% or less after 24 months at 25° C. and 60% RH. In another embodiment, described herein is a packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, wherein the fusidic acid, salt thereof, or a combination thereof degrades by about 9% or less after 24 months at 25° C. and 60% RH. A further embodiment of the above article is one wherein the composition further comprises mannitol. A further embodiment of any of the above articles is one wherein the container is an oxygen resistant container. A further embodiment of any of the above articles is one wherein the oxygen resistant container comprises oxygen resistant HDPE. A further embodiment of any of the above articles is one wherein the oxygen resistant container comprises a metal foil. A further embodiment of any of the above articles is one wherein the packaged dosage unit further comprises an insert comprising an antioxidant, where the insert is in the container. A further embodiment is one wherein the insert is a StabilOx™ Packet. A further embodiment of any of the above articles is one wherein the packaged dosage unit further comprises a reduced oxygen atmosphere, where the reduced oxygen atmosphere is in the container. A further embodiment is one wherein the reduced oxygen atmosphere comprises at least about 85% nitrogen. A further embodiment is one wherein the reduced oxygen atmosphere comprises at least about 90% nitrogen. A further embodiment is one wherein the reduced oxygen atmosphere comprises at least about 95% nitrogen. A further embodiment is one wherein the reduced oxygen atmosphere comprises at least about 98% nitrogen. A further embodiment is one wherein the reduced oxygen atmosphere comprises at least about 99% nitrogen.

As another aspect of the invention a method of treating a disease in a patient is described, the method comprising the step of administering to the patient a therapeutically effective amount of fusidic acid, or a pharmaceutically acceptable salt thereof, in a composition or one or more dosage units of any one of the embodiments described herein in which the fusidic acid component is protected from oxidation, including the preceding embodiments, where the disease is a bacterial infection. One embodiment of the method is a method of treating a disease in a patient, the method comprising the step of administering to the patient a therapeutically effective amount of fusidic acid, or a pharmaceutically acceptable salt thereof, in one or more dosage units of any one of the embodiments described herein in which the fusidic acid component is protected from oxidation, including the preceding embodiments, where the disease is a bacterial infection. In one embodiment of any of the above methods, the patient is a human.

In one embodiment with regard to the bacterial infection, the bacterial infection is an infection caused by bacteria selected from the group consisting of staphylococci, including coagulase-negative staphylococci and coagulase-positive staphylococci, streptococci, including Group A beta hemolytic streptococci, non-Group A beta hemolytic streptococci and viridans group streptococci, enterococci, Nesseria species, Clostridium species, Bordetella species, Bacillus species and Corynebacterium species. In one embodiment, the bacterial infection is an infection caused by bacteria selected from the group consisting of Staphylococcus aureus (methicillin-resistant and -susceptible), Staphylococus epidermidis, Staphylococus hemolyticus, Staphylococus saprophyticus, Staphylococus lugdunensis, Staphylococus capitis, Staphylococus caprae, Staphylococus saccharolyticus, Staphylococus simulans, Staphylococus warneri, Staphylococus hominis, Staphylococus intermedius, Staphylococcus pseudointermedius, Staphylococus lyricus, Streptococcus pyogenes, Streptococcus agalactiae, Streptococcus dysgalactiae subspecies dysgalactiae, Streptococcus anginosus, Streptococcus mitis, Streptococcus salivarius, Streptococcus bovis, Streptococcus mutans, Neisseria gonorrhoeae, Neisseria meningitidis, Bacillus anthracis, Bordetella pertussis, Clostridium difficile, Enterococcus faecalis, Enterococcus faecium and Corynebacterium diphtheriae. In another embodiment, the bacterial infection is an infection caused by Enterococcus faecalis or Enterococcus faecium.

In an additional embodiment with regard to the bacterial infection, the bacterial infection is an infection selected from the group consisting of a skin and soft tissue infection, a bone infection, a joint infection, pneumonia, a wound infection, a burn infection, an infection of the blood, and an infection associated with cystic fibrosis.

The treatment may be therapeutic treatment of a disease or may be in the form of antibiotic prophylaxis in a procedure such as a dental or surgical procedure, or as otherwise indicated.

Illustrative dosing protocols are described in co-pending U.S. Patent Application Publication No. 2011/0009375, the disclosure of which is incorporated herein in its entirety.

Another embodiment of any of the above methods further comprises the step of administering another antimicrobial, such as rifampicin.

In another embodiment, described herein is the pharmaceutical composition or dosage unit of any of the preceding embodiments wherein the per cent change in assay of the fusidic acid, or pharmaceutically acceptable salt thereof, is less than 5 per cent from the initial, normalized value following storage in an oxygen-resistant HDPE container at 25° C.±2° C. at 60%±5% relative humidity for 6 months.

In another embodiment, described herein is the pharmaceutical composition or dosage unit of any of the preceding embodiments wherein the per cent in assay of any degradation product of the fusidic acid, or pharmaceutically acceptable salt thereof, increases less than 2-fold, or 1.5-fold following storage in an oxygen-resistant HDPE container at 25° C.±2° C. at 60%±5% relative humidity for 6 months.

In another embodiment, described herein is the package of any one of the preceding embodiments wherein the per cent change in assay of the fusidic acid, or pharmaceutically acceptable salt thereof, is less than 5 per cent from the initial, normalized value following storage at 25° C.±2° C. at 60%±5% relative humidity for 6 months.

In another embodiment, described herein is the package of any one of the preceding embodiments wherein the per cent in assay of any degradation product of the fusidic acid, or pharmaceutically acceptable salt thereof, increases less than 2-fold, or 1.5-fold following storage at 25° C.±2° C. at 60%±5% relative humidity for 6 months.

Several illustrative embodiments of the invention are described by the following enumerated clauses:

1. A solid pharmaceutical composition comprising fusidic acid, or a pharmaceutically acceptable salt thereof, and mannitol.

2. A dosage unit comprising a solid pharmaceutical composition comprising fusidic acid, or a pharmaceutically acceptable salt thereof, and mannitol, where the fusidic acid or salt thereof is present in the range from about 275 mg to about 1,000 mg.

3. The dosage unit of clause 2 where the fusidic acid or salt thereof is present in the range from about 300 mg to about 900 mg.

4. The dosage unit any one of clauses 2 to 3 where the fusidic acid or salt thereof is present in the range from about 300 mg to about 800 mg.

5. The dosage unit any one of clauses 2 to 4 where the fusidic acid or salt thereof is present in the range from about 300 mg to about 700 mg.

6. The dosage unit any one of clauses 2 to 5 where the fusidic acid or salt thereof is present in the range from about 300 mg to about 600 mg.

7. The dosage unit any one of clauses 2 to 6s where the fusidic acid or salt thereof is present at about 300 mg.

8. The dosage unit of any one of clauses 2 to 6 where the fusidic acid or salt thereof is present at about 600 mg.

9. The pharmaceutical composition or dosage unit of any of the preceding clauses wherein the w/w ratio of the fusidic acid or salt thereof to mannitol is in the range from about 1:1 to about 10:1.

10. The pharmaceutical composition or dosage unit of any of the preceding clauses wherein the w/w ratio of the fusidic acid or salt thereof to mannitol is in the range from about 2:1 to about 5:1.

11. The pharmaceutical composition or dosage unit of any of the preceding clauses wherein the w/w ratio of the fusidic acid or salt thereof to mannitol is in the range from about 3:1 to about 4:1.

12. The pharmaceutical composition or dosage unit of any of the preceding clauses wherein the fusidic acid or salt thereof is present at about 10% to about 90% by weight.

13. The pharmaceutical composition or dosage unit of any of the preceding clauses wherein the fusidic acid or salt thereof is present at about 20% to about 80% by weight.

14. The pharmaceutical composition or dosage unit of any of the preceding clauses wherein the fusidic acid or salt thereof is present at about 30% to about 70% by weight.

15. The pharmaceutical composition or dosage unit of any of the preceding clauses wherein the fusidic acid or salt thereof is present at about 40% to about 60% by weight.

16. A packaged article comprising a container and the pharmaceutical composition or the dosage unit of any one of clauses 1 to 15 wherein the pharmaceutical composition or the dosage unit is inside the container and wherein the container is an oxygen resistant and/or oxygen impermeable container.

17. A packaged article comprising a container and the pharmaceutical composition or the dosage unit of any one of clauses 1 to 15 wherein the pharmaceutical composition or the dosage unit is inside the container and wherein the container or package includes an antioxidant compound or composition.

18. A packaged article comprising a container and the pharmaceutical composition or the dosage unit of any one of clauses 1 to 15 wherein the pharmaceutical composition or the dosage unit is inside the container and wherein the container or package includes an atmosphere with reduced oxygen or an atmosphere that is substantially free of oxygen.

19. The article of Claim 18 wherein the container or package includes a nitrogen atmosphere with reduced oxygen or substantially free of oxygen.

20. A packaged article comprising a container and the pharmaceutical composition or the dosage unit of any one of clauses 1 to 15 wherein the pharmaceutical composition or the dosage unit is inside the container and wherein:

a) the container is an oxygen resistant and/or oxygen impermeable container and the container or package includes an antioxidant compound or composition; or

b) the container is an oxygen resistant and/or oxygen impermeable container and the container or package includes an atmosphere with reduced oxygen or that is substantially free of oxygen; or

c) the container or package includes an antioxidant compound or composition and the container or package includes an atmosphere with reduced oxygen or that is substantially free of oxygen.

21. A packaged article comprising a container and the pharmaceutical composition or the dosage unit of any one of clauses 1 to 15 wherein the pharmaceutical composition or the dosage unit is inside the container and wherein the container is an oxygen resistant and/or oxygen impermeable container; the container or package includes an antioxidant compound or composition; and the container or package includes an atmosphere with reduced oxygen or that is substantially free of oxygen.

22. The pharmaceutical composition, dosage unit, or article of any one of the preceding clauses wherein the assay value of the fusidic acid or salt thereof is about 90% or more after 24 months at 25° C. and 60% RH.

23. The pharmaceutical composition, dosage unit, or article of any one of the preceding clauses wherein the assay value of the fusidic acid or salt thereof is about 91% or more after 24 months at 25° C. and 60% RH.

24. The pharmaceutical composition, dosage unit, or article of any one of the preceding clauses wherein the assay value of the fusidic acid or salt thereof is about 92% or more after 24 months at 25° C. and 60% RH.

25. The pharmaceutical composition, dosage unit, or article of any one of the preceding clauses wherein the assay value of the fusidic acid or salt thereof is about 93% or more after 24 months at 25° C. and 60% RH.

26. The pharmaceutical composition, dosage unit, or article of any one of the preceding clauses wherein the assay value of the fusidic acid or salt thereof is about 94% or more after 24 months at 25° C. and 60% RH.

27. The pharmaceutical composition, dosage unit, or article of any one of the preceding clauses wherein the assay value of the fusidic acid or salt thereof is about 95% or more after 24 months at 25° C. and 60% RH.

28. The pharmaceutical composition, dosage unit, or article of any one of the preceding clauses wherein the assay value of the fusidic acid or salt thereof is about 96% or more after 24 months at 25° C. and 60% RH.

29. The pharmaceutical composition, dosage unit, or article of any one of the preceding clauses wherein the assay value of the fusidic acid or salt thereof is about 97% or more after 24 months at 25° C. and 60% RH.

30. The pharmaceutical composition, dosage unit, or article of any one of the preceding clauses wherein the assay value of the fusidic acid or salt thereof is about 98% or more after 24 months at 25° C. and 60% RH.

31. The pharmaceutical composition, dosage unit, or article of any one of the preceding clauses wherein the assay value of the fusidic acid or salt thereof is about 99% or more after 24 months at 25° C. and 60% RH.

32. The pharmaceutical composition, dosage unit, or article of any one of the preceding clauses wherein the assay value of the fusidic acid or salt thereof is about 99.5% or more after 24 months at 25° C. and 60% RH.

33. The pharmaceutical composition, dosage unit, or article of any one of the preceding clauses wherein the assay value of the fusidic acid or salt thereof is about 90% or more after 12 months at 40° C. and 75% RH.

34. The pharmaceutical composition, dosage unit, or article of any one of the preceding clauses wherein the assay value of the fusidic acid or salt thereof is about 91% or more after 12 months at 40° C. and 75% RH.

35. The pharmaceutical composition, dosage unit, or article of any one of the preceding clauses wherein the assay value of the fusidic acid or salt thereof is about 92% or more after 12 months at 40° C. and 75% RH.

36. The pharmaceutical composition, dosage unit, or article of any one of the preceding clauses wherein the assay value of the fusidic acid or salt thereof is about 93% or more after 12 months at 40° C. and 75% RH.

37. The pharmaceutical composition, dosage unit, or article of any one of the preceding clauses wherein the assay value of the fusidic acid or salt thereof is about 94% or more after 12 months at 40° C. and 75% RH.

38. The pharmaceutical composition, dosage unit, or article of any one of the preceding clauses wherein the assay value of the fusidic acid or salt thereof is about 95% or more after 12 months at 40° C. and 75% RH.

39. The pharmaceutical composition, dosage unit, or article of any one of the preceding clauses wherein the assay value of the fusidic acid or salt thereof is about 96% or more after 12 months at 40° C. and 75% RH.

40. The pharmaceutical composition, dosage unit, or article of any one of the preceding clauses wherein the assay value of the fusidic acid or salt thereof is about 97% or more after 12 months at 40° C. and 75% RH.

41. The pharmaceutical composition, dosage unit, or article of any one of the preceding clauses wherein the assay value of the fusidic acid or salt thereof is about 98% or more after 12 months at 40° C. and 75% RH.

42. The pharmaceutical composition, dosage unit, or article of any one of the preceding clauses wherein the assay value of the fusidic acid or salt thereof is about 99% or more after 12 months at 40° C. and 75% RH.

43. The pharmaceutical composition, dosage unit, or article of any one of the preceding clauses wherein the assay value of the fusidic acid or salt thereof is about 99.5% or more after 12 months at 40° C. and 75% RH.

44. The pharmaceutical composition, dosage unit, or article of any one of the preceding clauses wherein the fusidic acid or salt thereof degrades by about 5% or less after 6 months at 25° C. and 60% RH.

45. The pharmaceutical composition, dosage unit, or article of any one of the preceding clauses wherein the fusidic acid or salt thereof degrades by about 5% or less after 6 months at 40° C. and 75% RH.

46. The pharmaceutical composition, dosage unit, or article of any one of the preceding clauses wherein the fusidic acid or salt thereof degrades by about 4% or less after 6 months at 25° C. and 60% RH.

47. The pharmaceutical composition, dosage unit, or article of any one of the preceding clauses wherein the fusidic acid or salt thereof degrades by about 4% or less after 6 months at 40° C. and 75% RH.

48. The pharmaceutical composition, dosage unit, or article of any one of the preceding clauses wherein the fusidic acid or salt thereof degrades by about 3% or less after 6 months at 25° C. and 60% RH.

49. The pharmaceutical composition, dosage unit, or article of any one of the preceding clauses wherein the fusidic acid or salt thereof degrades by about 3% or less after 6 months at 40° C. and 75% RH.

50. The pharmaceutical composition, dosage unit, or article of any one of the preceding clauses wherein the fusidic acid or salt thereof degrades by about 2% or less after 6 months at 25° C. and 60% RH.

51. The pharmaceutical composition, dosage unit, or article of any one of the preceding clauses wherein the fusidic acid or salt thereof degrades by about 2% or less after 6 months at 40° C. and 75% RH.

52. The pharmaceutical composition, dosage unit, or article of any one of the preceding clauses wherein the fusidic acid or salt thereof degrades by about 1% or less after 6 months at 25° C. and 60% RH.

53. The pharmaceutical composition, dosage unit, or article of any one of the preceding clauses wherein the fusidic acid or salt thereof degrades by about 1% or less after 6 months at 40° C. and 75% RH.

54. The pharmaceutical composition, dosage unit, or article of any one of the preceding clauses wherein the fusidic acid or salt thereof degrades by about 5% or less after 6 months at 25° C. and 60% RH.

55. The pharmaceutical composition, dosage unit, or article of any one of the preceding clauses wherein the fusidic acid or salt thereof degrades by about 5% or less after 6 months at 40° C. and 75% RH.

56. The pharmaceutical composition, dosage unit, or article of any one of the preceding clauses wherein the amount of 27-oxofusidic acid increases by less than about 2-fold after prolonged storage under ambient conditions, and/or after 6 months at 25° C. and 60% RH.

57. The pharmaceutical composition, dosage unit, or article of any one of the preceding clauses wherein the amount of 27-oxofusidic acid increases by less than about 50% after prolonged storage under ambient conditions, and/or after 6 months at 25° C. and 60% RH.

58. The pharmaceutical composition, dosage unit, or article of any one of the preceding clauses wherein the amount of 27-oxofusidic acid increases by less than about 25% after prolonged storage under ambient conditions, and/or after 6 months at 25° C. and 60% RH. 32. The pharmaceutical composition, dosage unit, or article of any one of the preceding clauses wherein the total amount of 3-ketofusidic acid, or the total amount of the sum of 3-ketofusidic acid and 11-ketofusidic acid increases by less than about 2-fold after prolonged storage under ambient conditions, and/or after 6 months at 25° C. and 60% RH.

59. The pharmaceutical composition, dosage unit, or article of any one of the preceding clauses wherein the total amount of 3-ketofusidic acid, or the total amount of the sum of 3-ketofusidic acid and 11-ketofusidic acid increases by less than about 50%, less than about 25%, less than about 10%, or less than about 5% after prolonged storage under ambient conditions, and/or after 12 months at 25° C. and 60% RH.

60. The pharmaceutical composition, dosage unit, or article of any one of the preceding clauses wherein the total amount of 3-ketofusidic acid, or the total amount of the sum of 3-ketofusidic acid and 11-ketofusidic acid increases by less than about 50%, less than about 25%, less than about 10%, or less than about 5% after prolonged storage under ambient conditions, and/or after 6 months at 40° C. and 75% RH.

61. The pharmaceutical composition, dosage unit, or article of any one of the preceding clauses wherein the total amount of 16-desacetylfusidic acid-21,16-lactone increases by less than about 2-fold, less than about 50%, or less than about 25% after prolonged storage under ambient conditions, and/or after 9 months at 25° C. and 60% RH.

62. The pharmaceutical composition, dosage unit, or article of any one of the preceding clauses wherein the total amount of 16-desacetylfusidic acid-21,16-lactone increases by less than about 2-fold, or less than about 50% after prolonged storage under ambient conditions, and/or after 12 months at 25° C. and 60% RH.

63. The pharmaceutical composition, dosage unit, or article of any one of the preceding clauses wherein the total amount of epi-16-desacetylfusidic acid increases by less than about 200%, or less than about 150%, or less than about 100% after prolonged storage under ambient conditions, and/or after 12 months at 25° C. and 60% RH.

64. The pharmaceutical composition, dosage unit, or article of any one of the preceding clauses wherein the total amount of 27-oxofusidic acid increases by less than about 50%, or less than about 25%, or less than about 10%, or less than about 5% after prolonged storage under ambient conditions, and/or after 12 months at 25° C. and 60% RH.

65. The pharmaceutical composition, dosage unit, or article of any one of the preceding clauses wherein the total amount of 27-oxofusidic acid increases by less than about 50%, or less than about 25%, or less than about 10%, or less than about 5% after prolonged storage under ambient conditions, and/or after 6 months at 40° C. and 75% RH.

66. A packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, or the dosage unit or the pharmaceutical composition described in any one of the preceding clauses, where the fusidic acid, salt thereof, or a combination thereof degrades by about 10% or less after 24 months at 25° C. and 60% RH.

67. A packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, or the dosage unit or the pharmaceutical composition described in any one of the preceding clauses, where the fusidic acid, salt thereof, or a combination thereof degrades by about 10% or less after 18 months at 25° C. and 60% RH.

68. A packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, or the dosage unit or the pharmaceutical composition described in any one of the preceding clauses, where the fusidic acid, salt thereof, or a combination thereof degrades by about 10% or less after 12 months at 40° C. and 75% RH.

69. A packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, or the dosage unit or the pharmaceutical composition described in any one of the preceding clauses, where the fusidic acid, salt thereof, or a combination thereof degrades by about 10% or less after 9 months at 40° C. and 75% RH.

70. A packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, or the dosage unit or the pharmaceutical composition described in any one of the preceding clauses, where the fusidic acid, salt thereof, or a combination thereof degrades by about 10% or less after 6 months at 40° C. and 75% RH.

71. The article of any one of clauses 66 to 70 wherein the composition further comprises mannitol.

72. The article of any one of clauses 66 to 70 wherein the container is an oxygen resistant container.

73. The article of clause 72 wherein the oxygen resistant container comprises oxygen resistant HDPE.

74. The article of clause 72 wherein the oxygen resistant container comprises a polymer film attached to a metal foil.

75. The article of any one of any one of clauses 66 to 74 wherein the packaged dosage unit further comprises an insert comprising an antioxidant, where the insert is inside the container.

76. The pharmaceutical composition, dosage unit, or article of any one of the preceding clauses wherein the antioxidant is a StabilOx™ Packet

77. The article of clause 75 wherein the insert is an iron-containing absorbent.

78. The article of any one of 66 to 77 wherein the packaged dosage unit further comprises a reduced oxygen atmosphere, wherein the reduced oxygen atmosphere is inside the container.

79. The article of clause 78 wherein the reduced oxygen atmosphere comprises at least about 85% nitrogen.

80. The article of clause 78 wherein the reduced oxygen atmosphere comprises at least about 90% nitrogen.

81. The article of Claim 40 wherein the reduced oxygen atmosphere comprises at least about 95% nitrogen.

82. The article of clause 78 wherein the reduced oxygen atmosphere comprises at least about 98% nitrogen.

83. The article of clause 78 wherein the reduced oxygen atmosphere comprises at least about 99% nitrogen.

84. The article of clause 78 herein the reduced oxygen atmosphere comprises a reduced pressure in the container.

85. A method of treating a disease in a host animal, the method comprising the step of administering to the host animal a therapeutically effective amount of fusidic acid, or a pharmaceutically acceptable salt thereof, in a composition or one or more dosage units of any one of the preceding clauses, where the disease is a bacterial infection.

86. A method of treating a disease in a host animal, the method comprising the step of administering to the host animal a therapeutically effective amount of fusidic acid, or a pharmaceutically acceptable salt thereof, in one or more dosage units of any one of clauses 2-15, where the disease is a bacterial infection.

87. The method of clause 85 or 86 wherein the host animal is a human.

88. The method of any one of clauses 85 to 87 further comprising the step of administering another antibacterial compound or composition.

Additional illustrative aspects and embodiments of the invention are further illustrated in the following non-limiting examples, in which fusidic acid denotes the sodium salt, sodium fusidate, as the active pharmaceutical ingredient (API); and percentages are on a weight:weight (w/w) basis.

### EXAMPLES

### Example

Fusidic Acid Formulations. Compositions comprising fusidic acid are prepared and characterized, as described in Table 1.

TABLE 1

| | | Disinte- | | |
|---|---|---|---|---|
| For- | | gration | | |
| mu- | | Time | Hardness | Friability |
| lation | Composition | (min) | (kP) | (% loss) |
| 44F | Intragranular: | 17-22 | 13-16 | 0.2-0.5 |
| | fusidate sodium (60%) | | | |
| | microcrystalline cellulose (15%) | | | |
| | sodium starch glycolate (4%) | | | |
| | magnesium stearate, NF (0.5%) | | | |
| | colloidal silicon dioxide, NF (0.5%) | | | |
| | Extragranular: | | | |
| | microcrystalline cellulose (15%) | | | |
| | sodium starch glycolate (4%) | | | |
| | magnesium stearate, NF (0.5%) | | | |
| | colloidal silicon dioxide, NF (0.5%) | | | |
| 44I | Intragranular: | 17-22 | 13-16 | 0.2-0.5 |
| | fusidate sodium (60%) | | | |
| | microcrystalline cellulose (15%) | | | |
| | sodium starch glycolate (4%) | | | |
| | magnesium stearate, NF (0.5%) | | | |
| | colloidal silicon dioxide, NF (0.5%) | | | |
| | Extragranular: | | | |
| | mannitol 300 DC (15%) | | | |
| | sodium starch glycolate (4%) | | | |
| | magnesium stearate, NF (0.5%) | | | |
| | colloidal silicon dioxide, NF (0.5%) | | | |

In the foregoing formulations, it is to be understood that various components may be altered to improve production capability and/or performance, such as including a total amount of magnesium stearate in the range from about 1% to about 8%, and/or including a total amount of colloidal silicon dioxide in the range from about 1% to about 2%.

### Example

Tablet Preparations and Package Configurations. 300 mg and 600 mg tablets are prepared from the above formulations using conventional roller compaction, then spray coated with Opadry White (4%, as an approximate 20% solids suspension). Briefly, the intragranular ingredients are sifted, then blended in a V-shell blender for 10 minutes. The blend is granulated using a roller compactor at a roller speed, roller hydraulic pressure, and granulator speed to provide a stream of brittle ribbon formation. The ribbon is milled and sifted to provide the final intragranular granulation. The extragranular ingredients are sifted, then blended with the intragranular granulation in a V-shell blender for 10 minutes. The final blend is placed in the hopper press, such as a Minipress II, and set for desired tablet weight, such as 1000 mg. Tablets have a % dissolution of 90-100% in 30 minutes, and approximately 100% in 45 minutes using conventional methods.

### Example

Bulk Storage Packages. The tablets (600 mg or 300 mg, 40 each) are placed in each Example package configuration, numbered 1-6 and shown in the following Table. All HDPE bottles were 75 cc with a CR cap. Each bottle included either a Tri-Sorb packets (0.5 g or 1 g) or a Stabilox™ packet. StabilOx™ is a commercially available oxygen and humidity management package.

| Example | Formulation | Tablet Size | Package |
|---|---|---|---|
| 1 | 44I | 600 mg | HDPE |
| 2 | 44I | 600 mg | Oxygen Resistance Bottle |
| 3 | 44F | 600 mg | HDPE |
| 4 | 44F | 600 mg | Oxygen Resistance Bottle |
| 5 | 44F | 300 mg | HDPE Plus Stabilox |
| 6 | 44F | 300 mg | HDPE |

### Example

Protocol for Identification, Assay and Impurity. HPLC analysis is performed with an Agilent 1100 HPLC System, variable wavelength (UV/VIS) detector at 235 nm, and Photo Diode Array (PDA) detector, Supelcosil, LC-18, 4.6×150 mm, 5 μm HPLC column, MeOH:10 g/L of H₃PO₄:H₂O:ACN, 6:23:23:48 by v/v/v/v mobile phase, 1.0 mL/minute, 60° C.±2° C.

The following impurity standards are independently prepared: sodium fusidate, 3-ketofusidic acid, 11-ketofusidic acid, and 16-desacetylfusidic acid, and optionally stored at refrigeration temperatures.

| | Relative Response | Relative Response |
|---|---|---|
| Name of Impurity | Time (RRT) | Factor (RRF) |
| 27-Oxofusidic acid (F) | 0.42 | 0.62 |
| 11-Ketofusidic Acid (H) | 0.71 | 0.71 |
| 3-Ketofusidic Acid (G) | 0.75 | 1.00 |
| 16-Desacetylfusidic Acid (O) | 1.60 | 0.72 |

Assay samples are prepared by weighing 20 CEM-102 tablets, grinding with a mortar and pestle, weighing and transferring an amount equivalent to 1 tablet weight into a 250 mL volumetric flask, adding about 150 mL of mobile phase, sonicating for about 45 to 60 minutes, and shaking using a mechanical shaker for about 15 minutes, diluting with additional mobile phase to 250 mL volume, mixing well, and filtering through a 0.45 μm nylon syringe filter. Illustrative Limit of Detection (LOD)=0.02%. Illustrative Limit of Quantitation (LOQ)=0.05%. It is to be understood that the detection and/or measurement of 16-desacetylfusidic acid may be made directly or as the corresponding 16-desacetylfusidic acid 21,16-lactone formed under acidic conditions, including acidic chromatographic conditions.

### Example

Protocol for Identification, Assay and Impurity. HPLC analysis is performed with an Agilent 1100 HPLC System, variable wavelength (UV/VIS) detector at 235 nm, and Photo Diode Array (PDA) detector, Waters Symmetry C-8, 4.6×150 mm, 3.5 μm HPLC column, MeOH:10 g/L of H₃PO₄:H₂O:ACN, 20:20:20:40 by v/v/v/v mobile phase, 1.0 mL/minute. The following illustrative gradient profile is used:

| Time (min) | % A | % B |
|---|---|---|
| 0.0 | 100 | 0 |
| 3.0 | 100 | 0 |
| 28.0 | 0 | 100 |
| 33.0 | 100 | 0 |
| 43.0 | 100 | 0 |

The following standards are used for impurity identification:

| Compound | RT (min.) | RRT |
|---|---|---|
| 27-oxofusidic acid | 12.5 | 0.62 |
| 3-ketofusidic acid | 16.0 | 0.80 |
| 11-ketofusidic acid | 16.5 | 0.82 |
| epi-16-desacetylfusidic acid | 18.6 | 0.93 |
| 16-desacetylfusidic acid 21,16-lactone | 24.9 | 1.24 |
| Sodium Fusidate | 20.0 | 1.00 |

### Examples

Stability Studies, Effects of Formulations and Packages. Bulk packaging of 100 count of 300 mg or 600 mg tablets and a 0.5 g Tri-Sorb® dehumidifying packet in white HDPE bottles with CR caps and an induction seal, or StabilitySolutions™ Bottles with CR caps are prepared. Packages are stored at (a) 25° C./60% relative humidity (RH) or (b) 40° C./75% RH for 6 months. API is assayed periodically during the storage period and reported in weight per cent API, as shown in the tables and figures below.

TABLE
600 mg Tablet Stability Results.

| | | | 25° C./60% RH (Months) | | | | |
|---|---|---|---|---|---|---|---|
| Example | Package Configuration | | Initial | 2 | 3 | 6 | 9 |
| 44I | HDPE | Assay (%) | 96.1 | 96.3 | 94.5 | 93.6 | 93.0 |
| | | Total | 0.83 | 0.99 | 0.89 | 1.65 | 1.95 |
| | | Impurities (%) | | | | | |
| | StabilitySolutions ™ | Assay (%) | 96.1 | 96.5 | 94.8 | 94.4 | 94.5 |
| | Bottle | Total | 0.83 | 1.02 | 0.90 | 1.36 | 1.29 |
| | | Impurities (%) | | | | | |
| 44F | HDPE | Assay (%) | 97.1 | 96.5 | 94.4 | 94.1 | 92.7 |
| | | Total | 0.78 | 1.61 | 1.00 | 1.86 | 2.10 |
| | | Impurities (%) | | | | | |
| | StabilitySolutions ™ | Assay (%) | 97.1 | 96.4 | 94.2 | 94.7 | 94.1 |
| | Bottle | Total | 0.78 | 1.13 | 0.97 | 1.5 | 1.38 |
| | | Impurities (%) | | | | | |
| | | | 40° C./75% RH (Months) | | | | |
| Example | Package Configuration | | Initial | 1 | 2 | 3 | 6 |
| 44I | HDPE | Assay (%) | 96.0 | 94.0 | 94.6 | 93.5 | 93.3 |
| | | Total | 0.83 | 1.35 | 1.39 | 1.45 | 3.06 |
| | | Impurities (%) | | | | | |
| | StabilitySolutions ™ | Assay (%) | 96.0 | 94.5 | 95.8 | ND | 93.9 |
| | Bottle | Total | 0.83 | 1.35 | 1.19 | ND | 1.69 |
| | | Impurities (%) | | | | | |
| 44F | HDPE | Assay (%) | 97.1 | 94.1 | 94.5 | 93.3 | 91.7 |
| | | Total | 0.78 | 1.49 | 1.12 | 1.57 | 2.86 |
| | | Impurities (%) | | | | | |
| | StabilitySolutions ™ | Assay (%) | 97.1 | 94.6 | 94.2 | ND | 94.0 |
| | Bottle | Total | 0.78 | 1.45 | 1.39 | ND | 1.94 |
| | | Impurities (%) | | | | | |

The data show that both packaging configurations decrease the amount of and/or rate of degradation of fusidic acid, and salts thereof, compared to control. The data also show that formulations including mannitol exhibit an unexpected improvement in API stabilization compared to formulations that do not include mannitol.

TABLE
300 mg Tablet Stability Results; Formulation 44F.

| | | 25° C./60% RH | | | | |
|---|---|---|---|---|---|---|
| Package Configuration | | Initial | 1 | 3 | 6 | 9 |
| HDPE (Tri-Sorb ®) | Assay (%) | 96.9 | 96.5 | 95.0 | 91.8 | 89.0 |
| | Total | 0.85 | 0.98 | 2.30 | 1.75 | 2.71 |
| | Impurities (%) | | | | | |
| HDPE (Stabilox ™) | Assay (%) | 96.9 | 96.2 | 95.4 | 95.3 | 96.4 |
| | Total | 0.85 | 0.89 | 1.15 | 1.04 | 1.38 |
| | Impurities (%) | | | | | |

TABLE
300 mg Tablet Stability Results; Formulation 44F.

| | | 40° C./75% RH | | | |
|---|---|---|---|---|---|
| Package Configuration | | Initial | 1 | 3 | 6 |
| HDPE (Tri-Sorb ®) | Assay (%) | 96.9 | 94.6 | 92.9 | 90.1 |
| | Total | 0.85 | 1.64 | 2.68 | 2.97 |
| | Impurities (%) | | | | |
| HDPE (Stabilox ™) | Assay (%) | 96.9 | 96.9 | 94.4 | 93.1 |
| | Total | 0.85 | 1.08 | 1.56 | 1.81 |
| | Impurities (%) | | | | |

The data show that both packaging configurations decrease the amount of and/or rate of degradation of fusidic acid, and salts thereof, compared to control.

TABLE
Assay Data for Examples 5 and 6.

| Ex- | For- | | | | | | |
|---|---|---|---|---|---|---|---|
| am- | mula- | | Months | | | | |
| ple | tion | Container | Initial | 1 | 3 | 6 | 9 |
| 25° C./60% RH | | | | | | | |
| 5 | 44F | HDPE | 96.9% | 96.5% | 95.4% | 95.3% | 96.4% |
| | | and | | | | | |
| | | Stabilox | | | | | |
| 6 | 44F | HDPE | 96.9% | 96.5% | 95.0% | 91.8% | 89.0% |
| 40° C./75% RH | | | | | | | |
| 5 | 44F | HDPE | 96.9% | 96.9% | 94.4% | 93.1% | NA |
| | | and | | | | | |
| | | Stabilox | | | | | |
| 6 | 44F | HDPE | 96.9% | 94.6% | 92.9% | 90.1% | NA |

The data show that packaging configurations containing an oxygen absorbing insert decrease the amount of and/or rate of degradation of fusidic acid, and salts thereof, compared to control.

TABLE
Assay Data for Examples 4 and 3.

| Ex- | For- | | | | | | |
|---|---|---|---|---|---|---|---|
| am- | mula- | | | | | | |
| ple | tion | Container | Months | | | | |
| 25° C./60% RH | | | Initial | 2 | 3 | 6 | 9 |
| 4 | 44F | O₂ Re- | 97.5% | 96.4% | 94.2% | 94.7% | 94.1% |
| | | sistant | | | | | |
| | | HDPE | | | | | |
| 3 | 44F | HDPE | 97.5% | 96.5% | 94.4% | 94.1% | 92.7% |
| 40° C./75% RH | | | Initial | 1 | 2 | 3 | 6 |
| 4 | 44F | O₂ Re- | 97.5% | 94.6% | 94.2% | ND | 94.0% |
| | | sistant | | | | | |
| | | HDPE | | | | | |
| 3 | 44F | HDPE | 97.5% | 94.1% | 94.5% | 93.3% | 91.7% |

ND = Not Determined

The data show that packaging configurations including an oxygen resistant material decrease the amount of and/or rate of degradation of fusidic acid, and salts thereof, compared to control.

TABLE
Assay Data for Examples 2 and 3.

| Ex- | For- | | | | | | |
|---|---|---|---|---|---|---|---|
| am- | mula- | | | | | | |
| ple | tion | Container | Months | | | | |
| 25° C./60% RH | | | Initial | 2 | 3 | 6 | 9 |
| 3 | 44F | HDPE | 97.5% | 96.5% | 94.4% | 94.1% | 92.7% |
| 2 | 44I | O₂ Re- | 96.1% | 96.3% | 94.5% | 93.6% | 93.0% |
| | | sistant | | | | | |
| | | HDPE | | | | | |
| 40° C./75% RH | | | Initial | 1 | 2 | 3 | 6 |
| 3 | 44F | HDPE | 97.5% | 94.1% | 94.5% | 93.3% | 91.7% |
| 2 | 44I | O₂ Re- | 96.1% | 94.0% | 94.6% | 93.5% | 93.3% |
| | | sistant | | | | | |
| | | HDPE | | | | | |

The data show that packaging configurations including an oxygen resistant material decrease the amount of and/or rate of degradation of fusidic acid, and salts thereof, compared to control.

TABLE
Assay Data for Examples 1 and 2.

| Ex- | For- | | | | | | |
|---|---|---|---|---|---|---|---|
| am- | mula- | | | | | | |
| ple | tion | Container | Months | | | | |
| 25° C./60% RH | | | Initial | 2 | 3 | 6 | 9 |
| 1 | 44I | HDPE | 96.1% | 96.3% | 94.5% | 93.6% | 93.0% |
| 2 | 44I | O₂Re- | 96.1% | 96.5% | 94.8% | 94.4% | 94.5% |
| | | sistant | | | | | |
| | | HDPE | | | | | |
| 40° C./75% RH | | | Initial | 1 | 2 | 3 | 6 |
| 1 | 44I | HDPE | 96.1% | 94.0% | 94.6% | 93.5% | 93.3% |
| 2 | 44I | O₂Re- | 96.1% | 94.5% | 95.8% | ND | 93.9% |
| | | sistant | | | | | |
| | | HDPE | | | | | |

The data show that packaging configurations including an oxygen resistant material decrease the amount of and/or rate of degradation of fusidic acid, and salts thereof, compared to control.

### Table

### API Assay Comparison

TABLE
Assay Data for Examples 3, 5, and 6.

| | | | Months | | | | | |
|---|---|---|---|---|---|---|---|---|
| Example | Formulation | Container | Initial | 1 | 2 | 3 | 6 | 9 |
| 3 | 44F | HDPE | 97.5% | ND | 96.5% | 94.4% | 94.1% | 92.7% |
| | 600 mg | | | | | | | |
| 6 | 44F | HDPE | 96.9% | 96.5% | ND | 95.0% | 91.8% | 89.0 |
| | 300 mg | | | | | | | |
| 5 | 44F | HDPE- | 96.9% | 96.2% | ND | 95.4% | 95.3 | 96.4 |
| | 300 mg | StabilOx | | | | | | |
| | | insert | | | | | | |

ND = Not Determined.

The data show that packaging configurations containing an oxygen absorbing insert decrease the amount of and/or rate of degradation of fusidic acid, and salts thereof, compared to control.

### Example

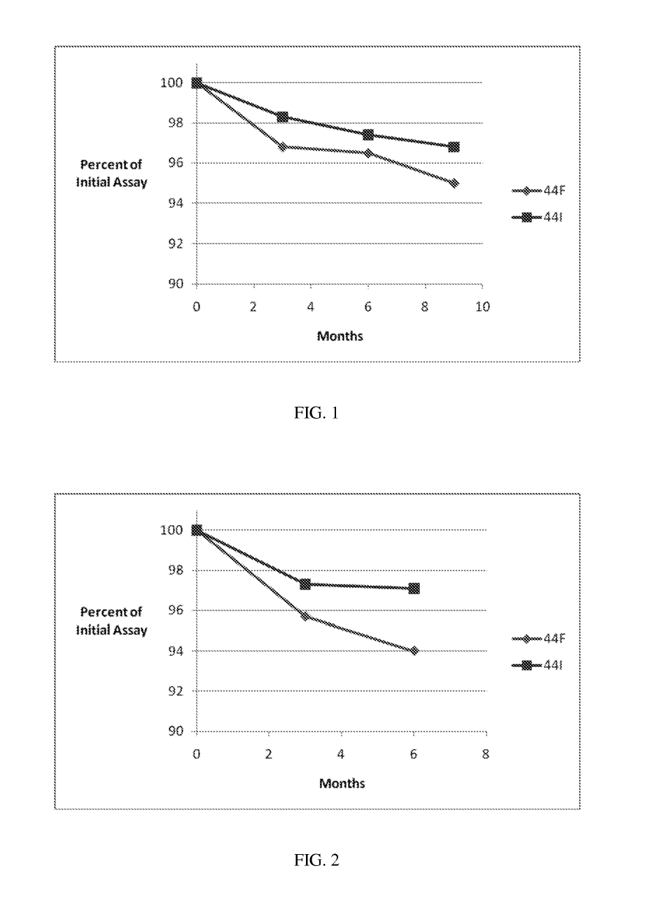

Comparison of Formulations and Packages. The API stability data of two formulations described herein are compared by looking at the change of the API assay value (Timepoint Assay−Time 0 Assay=Change in Assay) throughout the study. The data are normalized for the difference in starting percent of the fusidic acid in each tablet by comparing the percent API remaining of the Time Zero assay value over time, and shown in the following tables and in FIG. 1 and FIG. 2.

TABLE
Percent of Initial Assay of 600 mg
tablets in HDPE at 25° C./60% RH

| Ex- | | | | | | |
|---|---|---|---|---|---|---|
| am- | Formu- | | | | | |
| ple | lation | Container | Initial | 3 mo | 6 mo | 9 mo |
| 3 | 44F | HDPE | 97.5 | 94.4 | 94.1 | 92.7% |
| | Percent of | | 100.0 | 96.8 | 96.5 | 95.0 |
| | Initial Assay | | | | | |
| 1 | 44I | HDPE | 96.1 | 94.5 | 93.6 | 93.0% |

| | | | | |
|---|---|---|---|---|
| Percent of | 100.0 | 98.3 | 97.4 | 96.8 |
| Initial Assay | | | | |

TABLE
Percent of Initial Assay of 600 mg tablets in HDPE at 40° C./75% RH

| Example | Formulation | Container | Time = 0 | 3 mo | 6 mo |
|---|---|---|---|---|---|
| 3 | 44F | HDPE | 97.5 | 93.3 | 91.7 |
| | Percent of Initial Assay | | 100.0 | 95.7 | 94.0 |
| 1 | 44I | HDPE | 96.1 | 93.5 | 93.3 |

| | | | |
|---|---|---|---|
| Percent of Initial Assay | 100.0 | 97.3 | 97.1 |

The data show that mannitol has a stabilizing effect on the formulation by decreasing the overall degradation of fusidic acid, and salts thereof in control containers.

TABLE
Percent of Initial Assay of 600 mg tablet
in O₂ Resistant HDPE at 25° C./60% RH

| Ex- | | | | | | |
|---|---|---|---|---|---|---|
| am- | Formu- | | | | | |
| ple | lation | Container | Time = 0 | 3 mo | 6 mo | 9 mo |
| 4 | 44F | O₂ Resistant | 97.5% | 94.2% | 94.7% | 94.1% |
| | | HDPE | | | | |
| | Percent of | | 100.0 | 96.6 | 97.1 | 96.5 |
| | Initial Assay | | | | | |
| 2 | 44I | O₂ Resistant | 96.1% | 94.8% | 94.4% | 94.5% |
| | | HDPE | | | | |

| | | | | |
|---|---|---|---|---|
| Percent of | 100.0 | 98.6 | 98.2 | 98.2 |
| Initial Assay | | | | |

TABLE
Percent of Initial Assay of 600 mg tablet
in O₂ Resistant HDPE at 40° C./75% RH

| Ex- | | | | | | |
|---|---|---|---|---|---|---|
| am- | Formu- | | | | | |
| ple | lation | Container | Initial | 1 mo | 2 mo | 6 mo |
| 4 | 44F | O₂-resistant | 97.5 | 94.6 | 94.2 | 94.0 |
| | | HDPE | | | | |
| | Percent of | | 100.00 | 97.0 | 96.6 | 96.4 |
| | Initial Assay | | | | | |
| 2 | 44I | O₂-resistant | 96.1 | 94.5 | 95.8 | 93.9 |
| | | HDPE | | | | |

| | | | | |
|---|---|---|---|---|
| Percent of | 100.0 | 98.3 | 99.7 | 97.7 |
| Initial Assay | | | | |

The data show that further stabilization of the formulation by decreasing the overall degradation of fusidic acid, and salts thereof, is observed with mannitol formulations and packaging configurations described herein.

TABLE
Percent of Initial Assay of 300 mg tablet
in HDPE-Stabilox at 25° C./60% RH

| Ex- | | | | | | | |
|---|---|---|---|---|---|---|---|
| am- | Formu- | | | | | | |
| ple | lation | Container | Initial | 1 mo | 3 mo | 6 mo | 9 mo |
| 6 | 44F | HDPE | 96.9% | 96.5% | 95.0% | 91.8% | 89.0 |
| | Percent of | | 100.0 | 99.6 | 98.0 | 94.7 | 91.8 |
| | Initial Assay | | | | | | |
| 5 | 44F | HDPE- | 96.9% | 96.2% | 95.4% | 95.3 | 96.4 |
| | | Stabilox | | | | | |

| | | | | | |
|---|---|---|---|---|---|
| Percent of | 100.0 | 99.3 | 98.5 | 98.3 | 99.5 |
| Initial Assay | | | | | |

The data show that packaging configurations containing an oxygen absorbing insert decrease the amount of and/or rate of degradation of fusidic acid, and salts thereof, compared to control.

### Example

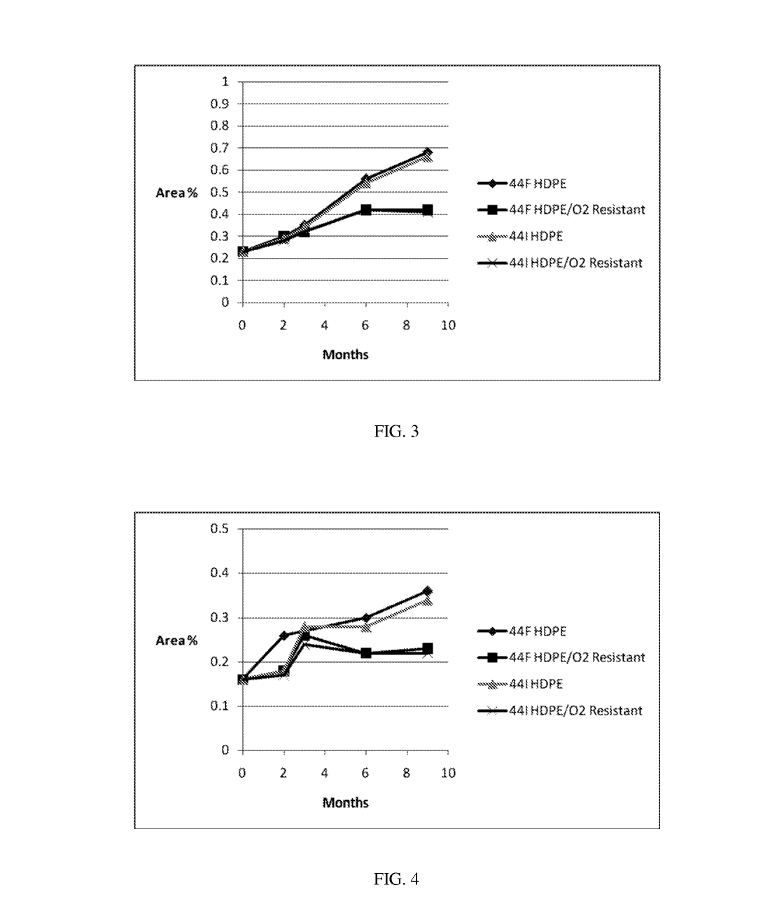

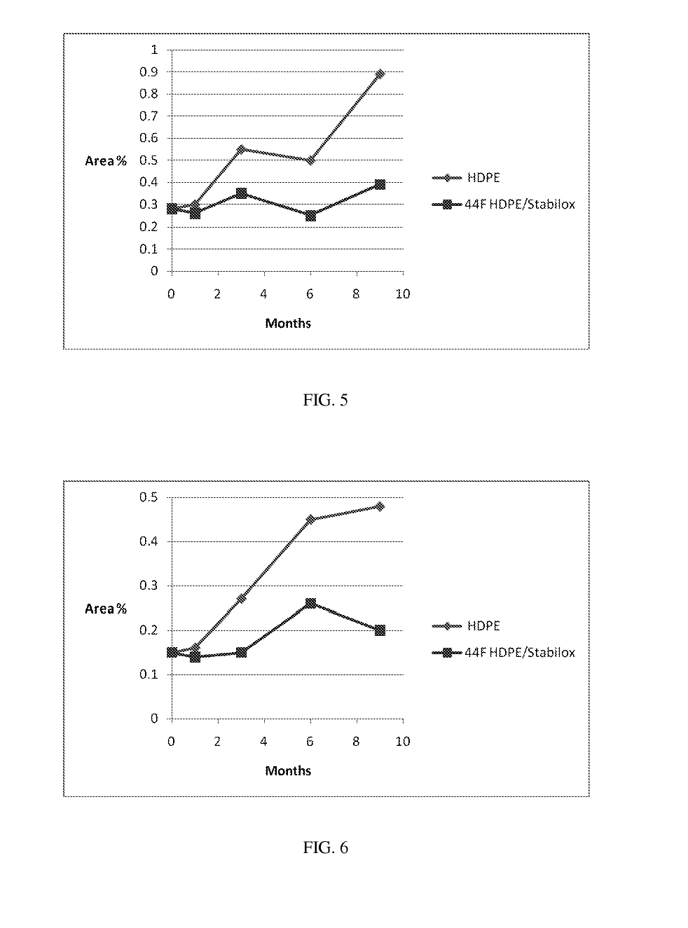

Evaluation of Impurities Levels and Changes During Storage in Bulk. The test articles were placed on stability at 25° C./60% RH for 12 months and at 40° C./75% RH for 6 months. Assay and related substances were measured at each pull point. Examples 1 to 6 were evaluated under various storage conditions, and were analyzed using the HPLC protocols described herein for increases in various impurities, including 27-oxofusidic acid (compound F), 11-ketofusidic acid (compound H), 3-ketofusidic acid (compound G), 16-desacetylfusidic acid (compound O), epi-16-desacetylfusidic acid (compound I), and 16-desacetylfusidic acid-21,16-lactone (compound K) during storage. The results in the following Table and in FIG. 3 and FIG. 4 demonstrate that the mannitol formulations and packaging configurations described herein substantially decrease the amount of impurity formation. FIG. 3 shows that 27-oxofusidic acid production is substantially decreased. FIG. 4 shows that 11-ketofusidic acid production and 3-ketofusidic acid production are both substantially decreased. 27-oxofusidic acid production, 11-ketofusidic acid production, and 3-ketofusidic acid production are also substantially decreased in Examples 5 compared to Example 6, when stored at 25° C./60% RH, as shown in FIG. 5 and FIG. 6.

| | | | | Assay | |
|---|---|---|---|---|---|
| Example | | | | 25° C./60% RH | 40° C./75% RH |
| Tablet | Formulation | Container | Measurement | (9 month) | (6 month) |
| 01 | 44I | HDPE | Change from initial | −3.1 | −2.8 |
| 600 mg | | Bottle | % increase/decrease | −3.23% | −2.91% |
| 02 | 44I | Oxygen | Change from initial | −1.6 | −2.2 |
| 600 mg | | Resistance | % increase/decrease | −1.66% | −2.29% |
| | | Bottle | | | |
| 03 | 44F | HDPE | Change from initial | −4.8 | −5.8 |
| 600 mg | | Bottle | % increase/decrease | −4.92% | −5.9% |
| 04 | 44F | Oxygen | Change from initial | −3.4 | −3.5 |
| 600 mg | | Resistance | % increase/decrease | −3.49% | −3.59% |
| | | Bottle | | | |
| 05 | 44F | HDPE | Change from initial | −0.5 | −3.7 |
| 300 mg | | Bottle | % increase/decrease | −0.52% | −3.82% |
| | | Plus | | | |
| | | Stabilox | | | |
| | | Packet | | | |
| 06 | 44F | HDPE | Change from initial | −7.9 | −6.8 |
| 300 mg | | Bottle | % increase/decrease | −81.5% | −7.02% |

| | | Total Impurities | |
|---|---|---|---|
| Example | | 25° C./60% RH | 40° C./75% RH |
| Tablet | Measurement | (9 month) | (6 month) |
| 01 | Change from initial | +1.12 | +2.23 |
| 600 mg | % increase/decrease | +135% | +269% |
| 02 | Change from initial | +0.46 | +0.81 |
| 600 mg | % increase/decrease | +38% | +98% |
| 03 | Change from initial | +1.32 | +2.08 |
| 600 mg | % increase/decrease | +169% | +267% |
| 04 | Change from initial | +0.60 | +1.16 |
| 600 mg | % increase/decrease | +77% | +149% |
| 05 | Change from initial | +0.53 | +0.96 |
| 300 mg | % increase/decrease | +62% | +113% |
| 06 | Change from initial | +1.86 | +2.12 |
| 300 mg | % increase/decrease | +219% | +249% |

| | | Oxidation Impurities | | | |
|---|---|---|---|---|---|
| | | 25° C./60% RH | | 40° C./75% RH | |
| Example | | (9 month) | | (6 month) | |
| Tablet | Measurement | Imp F | Imp G | Imp F | Imp G |
| 01 | Change from initial | +0.43 | +0.19 | +0.72 | +0.29 |
| 600 mg | % increase/decrease | +187% | +127% | +313% | +193% |
| 02 | Change from initial | +0.18 | +0.07 | +0.19 | +0.07 |
| 600 mg | % increase/decrease | +78% | +47% | +83% | +47% |
| 03 | Change from initial | +0.45 | +0.20 | +0.56 | +0.25 |
| 600 mg | % increase/decrease | +196% | +125% | +244% | +156% |
| 04 | Change from initial | +0.19 | +0.07 | +0.20 | +0.07 |
| 600 mg | % increase/decrease | +83% | +44% | +87% | +44% |
| 05 | Change from initial | +0.11 | +0.05 | +0.04 | +0.31 |
| 300 mg | % increase/decrease | +39% | +33% | +14% | +207% |
| 06 | Change from initial | +0.61 | +0.33 | +0.55 | +0.62 |
| 300 mg | % increase/decrease | +218% | +220% | +196% | +413% |

| | | Hydrolysis Impurities | |
|---|---|---|---|
| | | 25° C./60% RH | 40° C./75% RH |
| Example | | (9 month) | (6 month) |
| Tablet | Measurement | Imp K | Imp K |
| 01 | Change from initial | +0.02 | +0.11 |
| 600 mg | % increase/decrease | +22% | +122% |
| 02 | Change from initial | +0.03 | +0.14 |
| 600 mg | % increase/decrease | +33% | +155% |
| 03 | Change from initial | +0.10 | +0.27 |
| 600 mg | % increase/decrease | +166% | +450% |
| 04 | Change from initial | +0.10 | +0.30 |
| 600 mg | % increase/decrease | +166% | +500% |
| 05 | Change from initial | +0.09 | +0.38 |
| 300 mg | % increase/decrease | +129% | +543% |
| 06 | Change from initial | +0.09 | +0.38 |
| 300 mg | % increase/decrease | +129% | +543% |

### Example

Individual Storage Packages. The tablets (600 mg or 300 mg) are placed in each Example package configuration, numbered 7-15 and shown in the following Table. StabilOx™ is a commercially available oxygen and humidity management package. All other packaging is commercially available.

| Example | Material | Tablet |
|---|---|---|
| 07 | PVdC/250 PVC blister film with foil pouch overwrap with Stabilox packet | 600 |
| | | mg |
| 08 | PVdC/250 PVC blister film with foil pouch overwrap without Stabilox | 600 |
| | packet | mg |
| 09 | PVdC/250 PVC blister film without foil pouch overwrap | 600 |
| | | mg |
| 10 | PVdC/250 PVC blister film with foil pouch overwrap with Stabilox packet | 300 |
| | | mg |
| 11 | PVdC/250 PVC blister film with foil pouch overwrap without Stabilox | 300 |
| | packet | mg |
| 12 | PVdC/250 PVC blister film with foil pouch overwrap without Stabilox | 300 |
| | packet, nitrogen flushed. | mg |
| 13 | PVdC/250 PVC blister film without foil pouch overwrap. | 300 |
| | | mg |
| 14 | PVdC/250 PVC blister film without foil pouch overwrap, nitrogen flushed. | 300 |
| | | mg |
| 15 | Mono 250 PVC blister film with foil pouch overwrap with Stabilox packet | 300 |
| | | mg |

### Example

Evaluation of Impurities Levels and Changes During Storage in Blister Packs. The test articles were placed on stability at 25° C./60% RH for 12 months and at 40° C./75% RH for 6 months. Assay and related substances were measured at each pull point. Examples 7 to 15 were evaluated under various storage conditions, and were analyzed using the HPLC protocols described herein for increases in various impurities, including 27-oxofusidic acid (compound F), 11-ketofusidic acid (compound H), 3-ketofusidic acid (compound G), 16-desacetylfusidic acid (compound O), epi-16-desacetylfusidic acid (compound I), and 16-desacetylfusidic acid-21,16-lactone (compound K) during storage. The results in the following Table demonstrate that the mannitol formulations and packaging configurations described herein substantially decrease the amount of impurity formation.

| | | | Assay (%) | |
|---|---|---|---|---|
| Example | | | 25° C./60% RH | 40° C./75% RH |
| (Tablet) | Material | Measurement | (12 month) | (6 month) |
| 07 | PVdC/250 PVC blister | Change from initial | −3.8% | −3.1 |
| (600 mg) | film with foil pouch | % increase/decrease | −3.95 | −3.22 |
| | overwrap with Stabilox | | | |
| | packet | | | |
| 08 | PVdC/250 PVC blister | Change from initial | +0.6 | −1.1 |
| (600 mg) | film with foil pouch | % increase/decrease | +0.62 | −1.14 |
| | overwrap without Stabilox | | | |
| | packet | | | |
| 09 | PVdC/250 PVC blister | Change from initial | +0.1 | −1.7 |
| (600 mg) | film without foil pouch | % increase/decrease | +0.10 | −1.77 |
| | overwrap | | | |
| 10 | PVdC/250 PVC blister | Change from initial | +0.4 | −1.2 |
| (300 mg) | film with foil pouch | % increase/decrease | +0.42 | −1.25 |
| | overwrap with Stabilox | | | |
| | packet | | | |
| 11 | PVdC/250 PVC blister | Change from initial | +0.8 | −0.5 |
| (300 mg) | film with foil pouch | % increase/decrease | +0.83 | −0.52 |
| | overwrap without Stabilox | | | |
| | packet | | | |
| 12 | PVdC/250 PVC blister | Change from initial | +0.5 | −1.1 |
| (300 mg) | film with foil pouch | % increase/decrease | +0.52 | −1.14 |
| | overwrap without Stabilox | | | |
| | packet, nitrogen flushed. | | | |
| 13 | PVdC/250 PVC blister | Change from initial | +1.0 | −0.4 |
| (300 mg) | film without foil pouch | % increase/decrease | +1.04 | −0.42 |
| | overwrap. | | | |
| 14 | PVdC/250 PVC blister | Change from initial | +0.9 | −0.6 |
| (300 mg) | film without foil pouch | % increase/decrease | +0.94 | −0.63 |
| | overwrap, nitrogen | | | |
| | flushed. | | | |
| 15 | Mono 250 PVC blister | Change from initial | −2.3 | −3.0 |
| (300 mg) | film with foil pouch | % increase/decrease | −2.38 | −3.12 |
| | overwrap with Stabilox | | | |
| | packet | | | |

| | | Total Impurities (%) | |
|---|---|---|---|
| Example | | 25° C./60% RH | 40° C./75% RH |
| (Tablet) | Measurement | (12 month) | (6 month) |
| 07 | Change from initial | +1.4 | +1.6 |
| (600 mg) | % increase/decrease | +89 | +100 |
| 08 | Change from initial | −0.2 | +0.4 |
| (600 mg) | % increase/decrease | −13 | +25 |
| 09 | Change from initial | 0.0 | +0.6 |
| (600 mg) | % increase/decrease | 0 | +40 |
| 10 | Change from initial | +0.2 | +0.6 |
| (300 mg) | % increase/decrease | 13 | +40 |
| 11 | Change from initial | −0.1 | +0.4 |
| (300 mg) | % increase/decrease | −6 | +25 |
| 12 | Change from initial | +0.2 | +0.7 |
| (300 mg) | % increase/decrease | +18 | +64 |
| 13 | Change from initial | 0.0 | +0.7 |
| (300 mg) | % increase/decrease | 0 | +47 |
| 14 | Change from initial | −0.2 | +0.4 |
| (300 mg) | % increase/decrease | −13 | +26 |
| 15 | Change from initial | +1.8 | +2.1 |
| (300 mg) | % increase/decrease | +120 | +140 |

| | | Oxidation Impurities | | | |
|---|---|---|---|---|---|
| | | 25° C./60% | | 40° C./75% | |
| | | RH (%) | | RH (%) | |
| Example | | (12 month) | | (6 month) | |
| (Tablet) | Measurement | Imp F | Imp G | Imp F | Imp G |
| 07 | Change from initial | +0.42 | +0.27 | +0.30 | +0.22 |
| (600 mg) | % increase/decrease | +168 | +180 | +120 | +147 |
| 08 | Change from initial | 0.00 | +0.02 | −0.02 | +0.02 |
| (600 mg) | % increase/decrease | 0 | +14 | −8 | +14 |
| 09 | Change from initial | 0.00 | +0.01 | −0.02 | +0.02 |
| (600 mg) | % increase/decrease | 0 | +7 | _8 | +14 |
| 10 | Change from initial | +0.03 | +0.02 | 0.01 | +0.02 |
| (300 mg) | % increase/decrease | +13 | +14 | +4 | +14 |
| 11 | Change from initial | +0.03 | +0.02 | 0.00 | +0.02 |
| (300 mg) | % increase/decrease | +12 | +14 | 0 | +14 |
| 12 | Change from initial | +0.01 | +0.01 | 0.00 | +0.01 |
| (300 mg) | % increase/decrease | +6 | +9 | 0 | +9 |
| 13 | Change from initial | +0.02 | +0.02 | 0.00 | +0.03 |
| (300 mg) | % increase/decrease | +8 | +14 | 0 | =21 |
| 14 | Change from initial | −0.05 | −0.03 | −0.08 | −0.02 |
| (300 mg) | % increase/decrease | −20 | +21 | −32 | +14 |
| 15 | Change from initial | +0.50 | +0.21 | 0.41 | 0.28 |
| (300 mg) | % increase/decrease | +200 | +150 | +164 | +200 |

| | | Hydrolysis Impurities | | | |
|---|---|---|---|---|---|
| | | 25° C./60% | | 40° C./75% | |
| | | RH (%) | | RH (%) | |
| Example | | (12 month) | | (6 month) | |
| (Tablet) | Measurement | Imp I | Imp K | Imp I | Imp K |
| 07 | Change from initial | +0.10 | +0.06 | +0.29 | +0.30 |
| (600 mg) | % increase/decrease | +127 | +43 | +362 | +214 |
| 08 | Change from initial | +0.11 | +0.07 | +0.31 | +0.32 |
| (600 mg) | % increase/decrease | +138 | +50 | +388 | +229 |
| 09 | Change from initial | +0.12 | +0.09 | +0.31 | +0.47 |
| (600 mg) | % increase/decrease | +150 | +64 | +388 | +300 |
| 10 | Change from initial | +0.14 | +0.12 | +0.32 | +0.40 |
| (300 mg) | % increase/decrease | +175 | +92 | +400 | +308 |
| 11 | Change from initial | +0.12 | +0.14 | +0.31 | +0.39 |
| (300 mg) | % increase/decrease | +150 | +108 | +388 | +243 |
| 12 | Change from initial | +0.14 | +0.12 | +0.34 | +0.39 |
| (300 mg) | % increase/decrease | +175 | +80 | +425 | +260 |
| 13 | Change from initial | +0.14 | +0.15 | +0.31 | +0.62 |
| (300 mg) | % increase/decrease | +14 | +125 | +443 | +517 |
| 14 | Change from initial | +0.15 | +0.15 | +0.31 | +0.62 |
| (300 mg) | % increase/decrease | +214 | +125 | +442 | +250 |
| 15 | Change from initial | +0.11 | +0.14 | +0.29 | +0.40 |
| (300 mg) | % increase/decrease | +143 | +117 | +414 | +333 |

1. A solid pharmaceutical composition comprising fusidic acid, or a pharmaceutically acceptable salt thereof, and mannitol.

2. The pharmaceutical composition of claim 1 wherein the w/w ratio of the fusidic acid or salt thereof to mannitol is in the range from about 1:1 to about 10:1.

3.-4. (canceled)

5. A dosage unit comprising the solid pharmaceutical composition of claim 1, where the fusidic acid or salt thereof is present in the range from about 275 mg to about 1,000 mg.

6.-9. (canceled)

10. The dosage unit of claim 5 wherein the w/w ratio of the fusidic acid or salt thereof to mannitol is in the range from about 1:1 to about 10:1.

11.-12. (canceled)

13. The dosage unit of claim 5 wherein the fusidic acid or salt thereof is present at about 10% to about 90% by weight.

14. The dosage unit of claim 5 wherein the fusidic acid or salt thereof is present at about 40% to about 60% by weight.

15. A packaged article comprising a container and the pharmaceutical composition of claim 1 wherein the pharmaceutical composition or the dosage unit is inside the container and wherein the container is an oxygen resistant and/or oxygen impermeable container.

16. A packaged article comprising a container and the pharmaceutical composition of claim 1 wherein the pharmaceutical composition or the dosage unit is inside the container and wherein the container or package includes an antioxidant compound or composition.

17. A packaged article comprising a container and the pharmaceutical composition of claim 1 wherein the pharmaceutical composition or the dosage unit is inside the container and wherein the container or package includes an atmosphere with reduced oxygen or an atmosphere that is substantially free of oxygen.

18. (canceled)

19. A packaged article comprising a container and a pharmaceutical composition or dosage unit, wherein the pharmaceutical composition or the dosage unit is inside the container, and wherein the pharmaceutical composition or the dosage unit comprises fusidic acid, or a pharmaceutically acceptable salt thereof, or a combination thereof, where the fusidic acid, salt thereof, or a combination thereof degrades by about 10% or less after 24 months at 25° C. and 60% RH.

20. The article of claim 19 wherein the assay value of the fusidic acid or salt thereof is about 90% or more after 24 months at 25° C. and 60% RH.

20.-25. (canceled)

26. The article of claim 19 wherein the assay value of the fusidic acid or salt thereof is about 90% or more after 12 months at 40° C. and 75% RH.

27.-31. (canceled)

32. The article of claim 19 wherein the amount of 27-oxofusidic acid increases by less than about 2-fold after prolonged storage under ambient conditions.

33.-34. (canceled)

35. The article of claim 19 wherein the amount of 3-ketofusidic acid increases by less than about 2-fold after prolonged storage under ambient conditions.

36.-37. (canceled)

38. The article of claim 19 wherein the amount of 16-desacetylfusidic acid-21,16-lactone increases by less than about 2-fold after prolonged storage under ambient conditions.

39.-40. (canceled)

41. The article of claim 19 wherein the amount of epi-16-desacetylfusidic acid increases by less than about 2-fold after prolonged storage under ambient conditions.

42.-45. (canceled)

46. The article of claim 19 wherein the composition further comprises mannitol.

47.-53. (canceled)

54. A packaged article comprising a container and a dosage unit, wherein the dosage unit is inside the container, and wherein the dosage unit comprises a pharmaceutical composition comprising fusidic acid, or a pharmaceutically acceptable salt thereof, or a combination thereof, where the fusidic acid, salt thereof, or a combination thereof degrades by about 10% or less after 18 months at 25° C. and 60% RH.

55. (canceled)

56. The article of claim 54 where the fusidic acid, salt thereof, or a combination thereof degrades by about 10% or less after 9 months at 40° C. and 75% RH.

57. (canceled)

58. A method for treating a bacterial infection in a host animal, the method comprising the step of administering to the host animal a therapeutically effective amount of the pharmaceutical composition of claim 1.

59.-61. (canceled)
